(19) United States
(12) Patent Application Publication
(10) Pub. No.: US 2022/0154169 A9
(43) Pub. Date: May 19, 2022

(54) MAGNETIC ASSEMBLY

(71) Applicant: ArcherDX, Inc., San Francisco, CA (US)

(72) Inventors: Joshua Stahl, Boulder, CO (US); Jason Myers, Boulder, CO (US)

(73) Assignee: ArcherDX, Inc., San Francisco, CA (US)

(21) Appl. No.: 16/336,348

(22) PCT Filed: Sep. 22, 2017
(86) PCT No.: PCT/US2017/053064

§ 371 Date: Mar. 25, 2019

Related U.S. Application Data
(63) Continuation-in-part of application No. PCT/US2017/051924, filed on Sep. 15, 2017.
Application No. PCT/US2017/051924 is a continuation-in-part of application No. PCT/US2017/051927, filed on Sep. 15, 2017.
(60) Provisional application No. 62/398,841, filed on Sep. 23, 2016.
(60) Provisional application No. 62/399,152, filed on Sep. 23, 2016.
(60) Provisional application No. 62/399,157, filed on Sep. 23, 2016.
(60) Provisional application No. 62/399,184, filed on Sep. 23, 2016.
(60) Provisional application No. 62/399,195, filed on Sep. 23, 2016.
(60) Provisional application No. 62/399,205, filed on Sep. 23, 2016.
(60) Provisional application No. 62/399,211, filed on Sep. 23, 2016.
(60) Provisional application No. 62/399,219, filed on Sep. 23, 2016.

(51) Int. Cl. C12N 15/10; C12Q 1/6806; B01L 3/00; B03C 1/01; B03C 1/28; B03C 1/30

(52) U.S. Cl. CPC C12N 15/1013; C12Q 1/6806; B01L 3/502761; B03C 1/01; B03C 1/288; B03C 2201/26; B01L 2200/0668; B01L 2300/0816; B01L 2400/043; B01L 2400/0644; B03C 2201/18; B03C 1/30

(57) ABSTRACT

An apparatus comprising a magnetic assembly and methods for operating the apparatus are provided. The magnetic assembly may be used to manipulate molecules in a liquid preparation, for example to isolate or separate the molecules from the liquid. The magnetic assembly may be used to wash and/or isolate nucleic acid molecules of interest from a liquid preparation.

### RELATED APPLICATIONS

This application claims priority under 35 U.S.C. § 119(e) to U.S. Provisional Patent Application Nos. 62/398,841, 62/399,152, 62/399,157, 62/399,184, 62/399,195, 62/399,205, 62/399,211, and 62/399,219, each of which was filed on Sep. 23, 2016, and claims priority under 35 U.S.C. §§ 120 and 365(c) to PCT International Application No. PCT/US2017/051924, which was filed on Sep. 15, 2017, and which claims priority under 35 U.S.C. § 119(e) to U.S. Provisional Patent Application No. 62/395,339, which was filed on Sep. 15, 2016, and to PCT International Application No. PCT/US2017/051927, which was filed on Sep. 15, 2017, and which claims priority under 35 U.S.C. § 119(e) to U.S. Provisional Patent Application No. 62/395,347, which was filed on Sep. 15, 2016, the entire contents of each of which applications are hereby incorporated by reference.

### TECHNICAL FIELD

The present invention generally relates to assemblies for manipulating samples in fluidic systems and related methods.

### BACKGROUND

Numerous approaches for processing nucleic acids have been developed. Such methods often included multiple enzymatic, purification, and preparative steps that make them laborious and prone to error, including errors associated with contamination, systematic user errors, and process biases. As a result, it is often difficult to execute such processes reliably and reproducibly, particularly when the processes are being conducted commercially, e.g., in a multiplex or high-throughput context.

### SUMMARY

The present patent application generally relates to systems and related methods for processing samples (e.g., nucleic acids) in fluidic systems. In some embodiments, magnetic assemblies are provided to assist in the manipulation and processing of samples (e.g., samples containing one or more magnetic components. In some embodiments, magnetic assemblies are provided to assist in the processing of molecules such as nucleic acids or proteins in liquid samples, for example in the presence of one or more magnetic particles (e.g., magnetic beads). In some embodiments, one or more magnetic assemblies are included in a system that comprises a plurality of vessels for receiving liquid samples. Magnetic assemblies can be used to manipulate samples within the vessels. In some embodiments, magnetic particles (e.g., magnetic beads) are included in the vessels and/or in the samples. In some embodiments, the system comprises cartridges including cassettes and/or microfluidic channels that facilitate automated processing of the liquid samples, for example for automated nucleic acid library preparation. In some embodiments, systems and related methods are provided for automated processing of nucleic acids (e.g., associated with magnetic particles) to produce material for next generation sequencing and/or other downstream analytical techniques. In some embodiments, electromagnets may be used for magnetic control. In some embodiments, an apparatus for performing a chemical process comprises one or more vessels (e.g., 2-1,000, 2-500, 2-100, 2-24, 6, 8, 12, 16, 24, 32, 48, 64 or other number of vessels), and a magnetic assembly positioned adjacent to the one or more vessels, the magnetic assembly comprising one or more retractable magnets, each of the one or more retractable magnets capable of moving between i) a deployed position that is sufficiently proximate to a plurality of magnetic particles disposed in a vessel to force a magnetic particle present in the vessel against a wall of the vessel and ii) a retracted position that is sufficiently distant from the plurality of magnetic particles to release them from the wall of the vessel.

In some embodiments, the apparatus comprises one or more actuators configured to translocate the one or more retractable magnets between the deployed position and the retracted position. In some embodiments, the actuators are independently controllable. In some embodiments, the magnets are independently controllable.

In some embodiments, the apparatus comprises a thermal assembly positioned adjacent to the one or more vessels, the thermal assembly comprising one or more thermal elements configured to heat or cool the vessels. In some embodiments, each thermal element is a thermal pin defining hollow portion in which one of the one or more retractable magnets is positioned. In some embodiments, the apparatus comprises one or more pelletier elements for heating or cooling the thermal elements.

In some embodiments, the magnetic assembly comprises 2-1,000, 2-500, 2-100, 2-24, 6, 8, 12, 16, 24, 32, 48, 64 or other number of retractable magnets. In some embodiments, the one or more retractable magnets are independently-controllable.

In some embodiments, the chemical process is a nucleic acid purification process.

In some embodiments, the apparatus comprises a liquid sample disposed in one or more vessels, the liquid sample in each vessel comprising a plurality of magnetic particles (e.g., magnetic beads) having bound molecules of interest (e.g., bound nucleic acid or protein molecules or a combination thereof).

Accordingly, in some embodiments, an apparatus comprising a magnetic assembly can be used to perform a process (e.g., to assist in the isolation or purification of a molecule of interest such as a nucleic acid). In some embodiments, when in operation the apparatus comprises one or more vessels, a liquid sample disposed in each vessel, a plurality of magnetic beads in each vessel, an analyte of interest bound to the magnetic beads in each vessel, and a magnetic assembly positioned adjacent to each vessel. In some embodiments, the magnetic assembly comprises one or more magnets, one or more linear actuators (e.g., independently controlled linear actuators) coupled to the one or more magnets, each of the one or more linear actuators capable of moving the one or more magnets to a deployed position sufficiently proximate to the plurality of magnetic beads to draw the plurality of magnetic beads to a wall of the vessel, and a retracted position. In some embodiments, the magnetic assembly comprises one or more magnet lifting apparatuses coupled to the one or more magnets (e.g., via the actuators), each of the one or more magnet lifting apparatuses capable of moving the one or more magnets to an extended position and a contracted position. In some embodiments, the apparatus further comprises a thermal (e.g., heating) assembly positioned adjacent to each vessel. In some embodiments, the thermal assembly comprises one or more thermal (e.g., heating) pins, wherein each of the thermal pins defines a hollow portion in which one of the one or more magnets is positioned, a thermal (e.g., heating) block defining one or more through holes, each through hole positioned to receive one of the one or more thermal pins therethrough, an insulating liner positioned to surround at least a portion of the thermal block, the insulating liner defining one or more through holes, each through hole positioned to receive one of the one or more thermal pins therethrough, a cartridge temperature regulator (e.g., heater) disposed proximate to the thermal block; and a thermistor disposed proximate to the thermal block.

In some embodiments, a method for performing a chemical process comprises introducing a liquid sample containing one or more analytes of interest (e.g., one or more nucleic acids, proteins, other molecules, or any combination thereof) to a vessel, and introducing a plurality of magnetic beads to the liquid sample in the vessel, wherein the magnetic beads are capable of binding to the analyte (e.g., nucleic acid) in the liquid sample. In some embodiments, the liquid sample is mixed to form a homogenous mixture comprising the plurality of magnetic beads (e.g., with bound analyte) and a remainder portion. In some embodiments, one or more retractable magnets are deployed into a position sufficiently proximate to the plurality of magnetic beads to draw the plurality of magnetic beads (and bound analyte) to a wall of the vessel proximate to the magnet(s). The remainder portion can then be removed from the vessel (e.g., air flow, aspiration, flow of a replacement liquid, or other technique, for example that can be implemented via a microfluidic device), leaving the magnetic beads (and associated analyte molecules) in the vessel. In some embodiments, the magnetic beads are rinsed in order to rinse the analyte (e.g., nucleic acid) that is bound to the beads. In some embodiments, the magnetic beads and bound analyte are rinsed with a solvent. In some embodiments, the solvent is ethanol. In some embodiments, an elution buffer is introduced into the vessel (e.g., after rinsing) to release the nucleic acid from the magnetic beads.

In some embodiments, the rinsing is done while the beads remain on the side of the vessel (due to the magnet(s) being in the deployed position). In some embodiments, the elution buffer is introduced into the vessel while the beads remain on the side of the vessel.

In some embodiments, the one or more retractable magnets are retracted to a position sufficiently distant from the plurality of magnetic beads to release the plurality of magnetic beads from the wall of the vessel before, during, or after rinsing. In some embodiments, the one or more retractable magnets are retracted to a position sufficiently distant from the plurality of magnetic beads to release the plurality of magnetic beads from the wall of the vessel before, during, or after elution.

In some embodiments, the one or more retractable magnets are deployed to a position sufficiently proximate to the plurality of magnetic beads after washing and/or elution to draw the plurality of magnetic beads to the wall of the vessel to provide a purified solution containing the analyte (e.g., nucleic acid). In some embodiments, the purified solution can then be removed (e.g., for further processing or analysis, for example for sequencing).

In some embodiments, the temperature of the liquid sample can be maintained or altered (e.g., heated or cooled) during the washing and/or elution steps, for example using the one or more thermal elements in the apparatus.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 is a schematic drawing of a nucleic acid library preparation workflow;

FIG. 2A is a drawing of a system for automated nucleic acid library preparation using a microfluidic cartridge;

FIG. 2B is a drawing showing internal components of a system for automated nucleic acid library preparation using a microfluidic cartridge;

FIG. 3 is a perspective view of a microfluidic cartridge bay assembly;

FIG. 4A is a top view of a microfluidic cartridge carrier assembly;

FIG. 4B is a perspective view of a microfluidic cartridge;

FIG. 5 is an exploded view of a microfluidic cartridge;

FIG. 6A is a perspective view of an integrated assembly according to one or more embodiments;

FIG. 6B is a cross-sectional view of an integrated heating assembly and magnetic assembly shown in FIG. 6A; and,

FIG. 6C is an exploded view of an integrated heating assembly and magnetic assembly shown in FIGS. 6A and 6B.

### DETAILED DESCRIPTION

Aspects of the present patent application relate to magnetic assemblies for fluid handling systems. Magnetic assemblies can be used to manipulate sample components, for example to assist in chemical and/or biological analyses. In some embodiments, magnetic assemblies are provided in a system that also includes one or more cassettes comprising vessels and/or microfluidic channels.

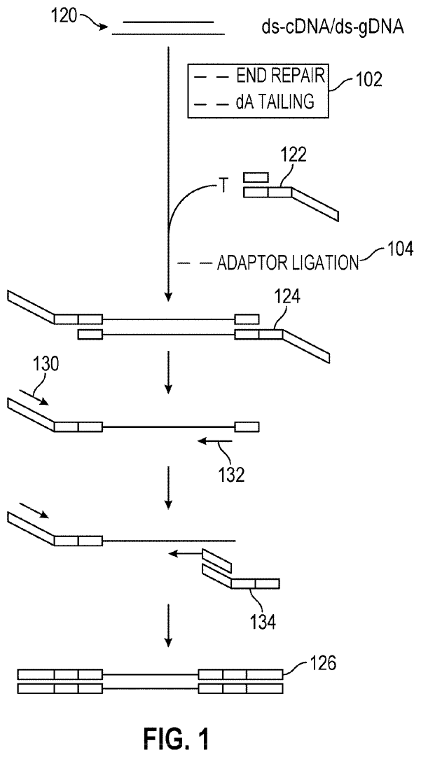

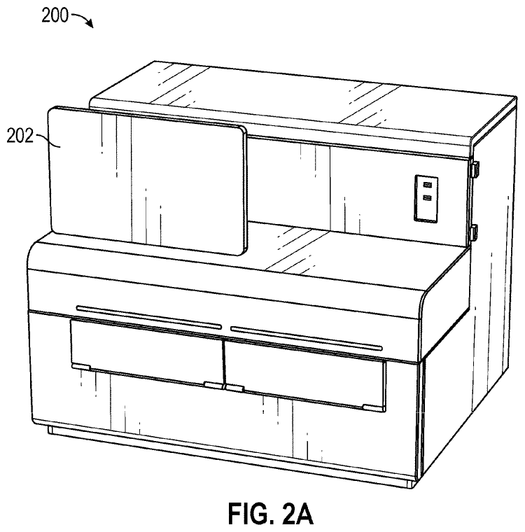

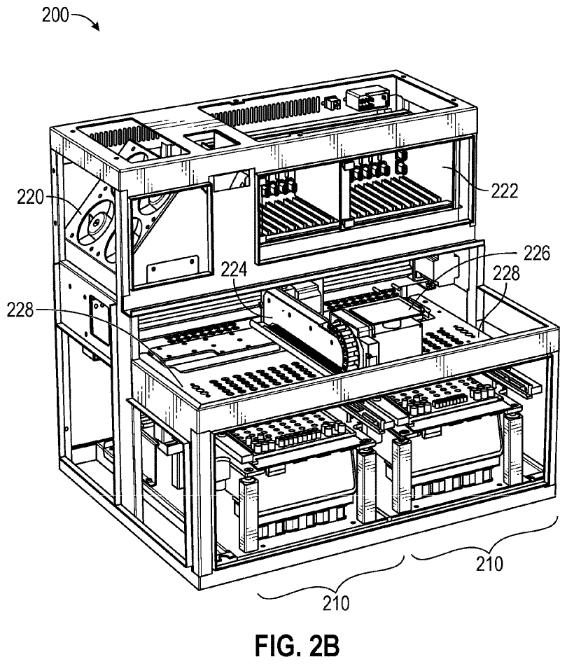

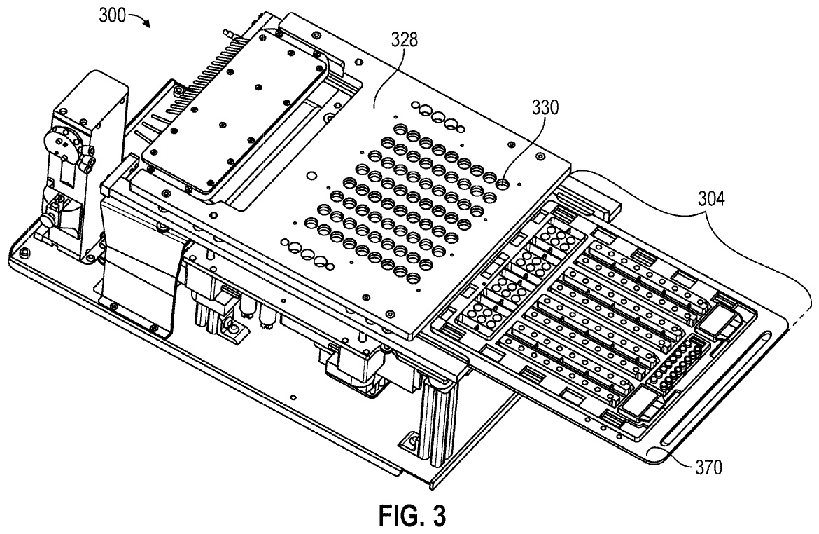

Magnetic assemblies can be used in the context of nucleic acid analyses to isolate or purify sample components and/or reaction products. Magnetic assemblies can be used, for example, to assist in the automation of a nucleic acid analysis (e.g., a nucleic acid analysis as illustrated in FIG. 1). Magnetic assemblies can be incorporated into an automated system (e.g., a system as illustrated in FIGS. 2A-2B). Magnetic assemblies can be configured to operate in a system comprising one or more microfluidic cartridges (e.g., one or more microfluidic cartridges illustrated in FIGS. 3, 4A-4B, and 5). In some embodiments, the magnetic assemblies may be incorporated into an assembly that also includes heating components that may be used in fluidic systems to form an integrated heating and magnetic assembly.

The magnetic assembly and/or integrated assembly may be used in methods for performing a chemical assay and/or for providing a purified solution.

The integrated assembly may comprise a first set of components for performing magnetic operations associated with chemical and/or biological analyses. This first set of components may be collectively referred to as a magnetic assembly. The magnetic assembly may comprise, for example, magnets, independently-controllable linear actuators for moving the magnets; and magnet lifting apparatuses. The integrated assembly may further comprise a second set of components for performing heating operations associated with performing chemical and/or biological analyses. This second set of components may be collectively referred to as a heating assembly. The heating assembly may comprise, for example, heating pins, a heating block, an insulating liner, a cartridge heater, and a thermistor. Some components may be shared between the two assemblies.

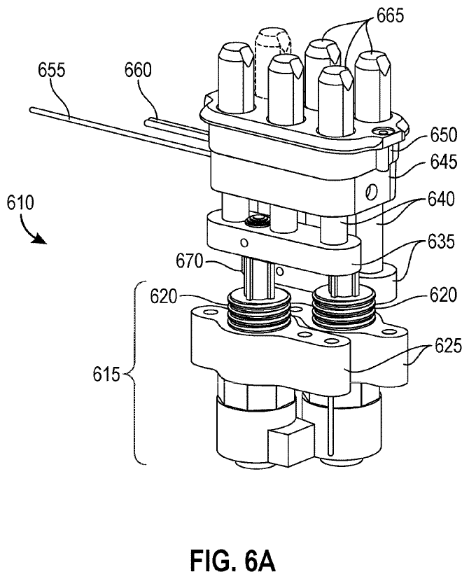

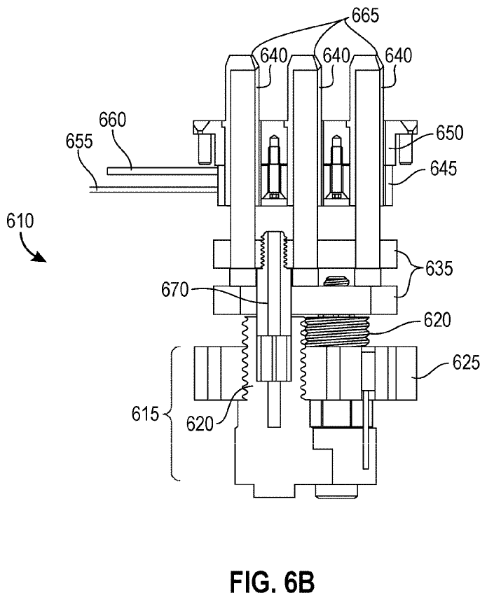

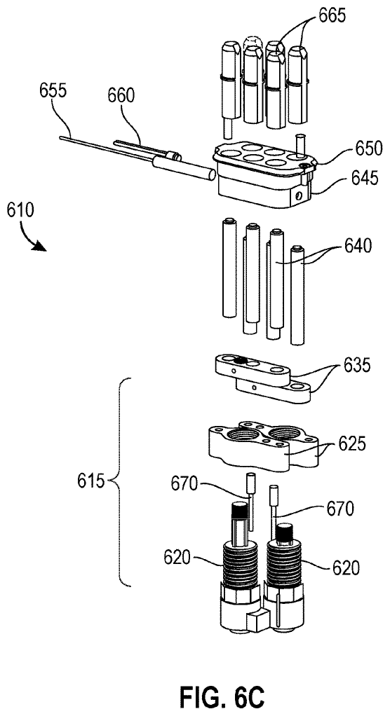

FIGS. 6A-6C illustrate different views of an integrated heating and magnetic assembly 610 according to one or more embodiments. FIG. 6A shows an integrated heating and magnetic assembly 610 according to a perspective view. FIG. 6B shows an integrated heating and magnetic assembly 610 according to a cross-sectional view. FIG. 6C shows an integrated heating and magnetic assembly 610 according to an exploded view.

In operation, the assembly 610 may be positioned proximate to a vessel or set of vessels that serves as a stage in performing chemical and/or biological analyses. During operation, fluid and a plurality of magnetic particles (e.g., magnetic beads) may be introduced into the vessel. Components of the fluid may bond and/or adhere to the magnetic particles. The particles may then be manipulated by the magnetic assembly to aid, for example, purifying a fluid for analysis. Such methods are discussed in more detail below.

The integrated magnetic and heating assembly 610 shown in FIGS. 6A-6C comprises independently-controllable linear actuators 615. Each actuator 615 is coupled to a set of magnets 640. The actuators 615 are capable during operation of moving the magnets 640 to a deployed position sufficiently proximate to a plurality of magnetic beads to draw the beads to a wall of the vessel Likewise, the actuators 615 are capable of moving the magnets 640 to a retracted position so that the magnetic beads are able to freely move in the vessel. Each actuator 615 comprises an actuator base 625 and an actuator rod 620 coupled to the actuator base. Threading on the actuator rod 620 aids in coupling the rod 620 to the base 625.

The assembly 610 further comprises magnetic lifting apparatus 635. Each lifter 635 is coupled to an actuator 615 through a threaded rod 670. The magnet lifting apparatuses 635 are also coupled to magnets 640. In operation, each lifter 635 is capable of moving the coupled magnets 640 to an extended position (e.g., more vertical position) and a contracted position (e.g., a lower position).

The integrated assembly 610 further comprises elements that form the heating assembly portion. The assembly includes, for example, heating pins 665, which are used for heating fluids in nearby vessels. As shown in FIGS. 6A-6C, each of the heating pins 665 defines a hollow portion in which one of the magnets is positioned. A heating block 645 defines through holes that receive each of the heating pins 665. An insulating liner 650 is positioned to surround at least a portion of the heating block 645. The insulating liner 650 also defines through holes positioned to receive the heating pins 665 therethrough. A cartridge heater 655 and a thermistor 660 are disposed proximate to the heating block.

While FIGS. 6A-6C show one specific embodiment of an integrated magnetic and heating assembly, it should be understood that alternative embodiments including different numbers of different components are understood to be within the scope of the disclosure.

The assembly described above may be used in systems that further include, according to certain embodiments, a cassette comprising a one or more vessels adapted and arranged to contain a fluid and/or reagent for performing a chemical and/or biological analysis. The vessel may be designed to have a particular shape or configuration, such as a tapered cross-sectional shape, e.g., to facilitate manipulation of a fluid and/or reagent within the vessel (e.g., a lyosphere). Channels connected to a channel system may be in fluid communication with the vessel. The channel system may be used to introduce and/or remove fluids and/or reagents into and from the vessel.

The assembly described above may be incorporated in methods for performing a chemical assay and/or providing a purified solution.

According to one embodiment of a such a method, a sample fluid (e.g., a liquid sample) containing an analyte of interest (e.g., a nucleic acid) is introduced to a vessel. A plurality of magnetic beads are also introduced to the vessel, at a volume based on the volume of sample fluid. The sample and bead solution are mixed thoroughly until the solution forms a homogenous mixture comprising magnetic beads with bound analyte (e.g., nucleic acid) and a remainder portion (e.g., the portion of the fluid not bound and/or adhering to the magnetic beads).

In some embodiments, magnetic particles can be made from synthetic polymers, porous glass, or metallic materials. In some embodiments, magnetic particles can incorporate and/or be coated with a magnetic material. In some embodiments, magnetic particles can be modified or coated with functional groups to attach one or more binding agents (e.g., to bind to a specific analyte of interest). However, in some embodiments the properties of the magnetic bead material (e.g., electric charge or electrostatic properties of the material) are sufficient to bind to the analyte of interest (e.g., positively charged to bind to negative charges on nucleic acid molecules).

Through use of an assembly described above, one or more retractably magnets are deploying into a position sufficiently proximate to the plurality of magnetic beads to draw the magnetic beads with bound nucleic acid to a wall of the vessel. While the beads are held to the wall of the vessel, the remainder portion of the sample fluid is removed (e.g., drained) from the vessel. The magnetic beads with bound nucleic acid are rinsed with a solvent (e.g., ethanol). The solvent is then removed to waste. The rinsing step may be repeated.

The magnets are then retracted from the vessel into a position sufficiently distant from the magnetic beads to release the magnetic beads from the wall of the vessel. Elution buffer is then introduced into the vessel and mixed thoroughly with the beads to free (or unbind) the nucleic acid from the magnetic beads. The retractable magnets are again deployed into a position sufficiently proximate to the plurality of magnetic beads to draw the plurality of magnetic beads to the wall of the vessel, leaving the purified solution containing nucleic acid remains in the vessel. The purified solution containing nucleic acid is then removed from the vessel.

Systems including cartridges with modular components (cassettes) and/or microfluidic channels for processing nucleic acids are generally provided. In some embodiments, systems and related methods are provided for automated processing of nucleic acids to produce material for next generation sequencing and/or other downstream analytical techniques. In some embodiments, systems described herein include a cartridge comprising, a frame, one or more cassettes which may be inserted into the frame, and a channel system for transporting fluids. In certain embodiments, the one or more cassettes comprise one or more reservoirs or vessels configured to contain and/or receive a fluid (e.g., a stored reagent, a sample). In some cases, the stored reagent may include one or more lyospheres. The systems and methods described herein may be useful for performing chemical and/or biological reactions including reactions for nucleic acid processing, including polymerase chain reactions (PCR). In some embodiments, systems and methods provided herein may be used for processing nucleic acids as depicted in FIG. 1. For example, in some embodiments, the nucleic acid preparation methods depicted in FIG. 1, which are described in greater detail herein, may be conducted in a multiplex fashion with multiple different (e.g., up to 8 different) samples being processed in parallel in an automated fashion. Such systems and methods may be implemented within a laboratory, clinical (e.g., hospital), or research setting.

In some embodiments, systems provided herein may be used for next generation sequencing (NGS) sample preparation (e.g., library sample preparation). In some embodiments, systems provided herein may be used for sample quality control. FIGS. 2A and 2B depict an example system 200 which serves as a laboratory bench top instrument which utilizes a number of disposable cassettes, primer cassettes, and bulk fluid cassettes. In some embodiments, this system is suitable for use on a standard laboratory workbench.

In some embodiments, a system may have a touch screen interface (e.g., as depicted in the exemplary system of FIG. 2A comprising a touch screen interface 202). In some embodiments, the interface displays the status of each of the one or more cartridge bays with “estimated time to complete”, “current process step”, or other indicators. In some embodiments, a log file or report may be created for each of the one or more cartridges. In some embodiments, the log file or report may be saved on the instrument. In some embodiments, a text file or output may be sent from the instrument, e.g., for a date range of cartridges processed or for a cartridge with a particular serial number.

In some embodiments, systems provided herein may comprise one or more cartridge bays (e.g., two, as depicted in the exemplary system of FIG. 2B comprising two cartridge bays 210), capable of receiving one or more nucleic acid preparation cartridges. In some embodiments, a space above the cartridge bay(s) is reserved for an XY positioner 224 to move an optics module 226 (and/or a barcode scanner, e.g., a 2-D barcode scanner) above lids 228 (e.g., heated lids) of each cartridge bay. In some embodiments, the system comprises an electronics module 222 that drives optics module 226 and XY positioner 224. In some embodiments, XY positioner 224 will position optics module 226 such that it can excite materials (e.g., fluorophores) in the vessel and collect the emitted fluorescent light. In some embodiments, this will occur through holes placed in the lid (e.g., heated lid) over each vessel. In some embodiments, a barcode scanner will confirm that appropriate cartridge and primer cassettes have been inserted in the system. In some embodiments, optics module 226 will collect light signals from each cartridge in each cartridge bay, as needed, during processing of a sample, e.g., during amplification of a nucleic acid to detect the level of the amplified nucleic acid. In some embodiments, the systems described herein comprise elements that assist in temperature regulation of components within the system, such as one or more fans or fan assemblies (e.g., the fan assembly 220 depicted in FIG. 2B).

In some embodiments, the one or more cartridge bays can process nucleic acid preparation cartridges, in any combination. In some embodiments, each cartridge bay is loaded, e.g., by the operator or by a robotic assembly. FIG. 3 depicts an exemplary drawing of a microfluidics cartridge bay assembly 300. In some embodiments, a cartridge is loaded into a bay when the bay is in the open position by placing the cartridge into a carrier plate 370 to form a carrier plate assembly 304. The carrier plate is itself, in some embodiments, a stand-alone component which may be removed from the cartridge bay. This cartridge bay holds the cartridge in a known position relative to the instrument. In some embodiments, a lid 328 (e.g., a heated lid) comprises one or more holes 330 to facilitate the processing and/or monitoring of reactions occurring in one or more vessels. In some embodiments, prior to loading a new cartridge onto the instrument, a primer cassette may be installed onto the cartridge. In some embodiments, the primer cassette would be packaged separately from the cartridge. In some embodiments, a primer cassette may be placed into a cartridge. In some embodiments, both primer cassettes and cartridges would be identified such that placing them onto the instrument allows the instrument to read them (e.g., using a barcode scanner) and initiate a protocol associated with the cassettes.

In some embodiments, prior to installing a carrier into the instrument, bulk reagents may be loaded into the carrier. In some embodiments, a user or robotic assembly may be informed as to which reagents to load and where to load them by the instrument or an interface on a remote sample loading station. In some embodiments, after loading a cartridge with a primer cassette into an instrument, a user would have the option of choosing certain reaction conditions (e.g., a number of PCR cycles) and/or the quantity of samples to be run on the cartridge. In some embodiments, each cartridge may have a capacity of 1, 2, 3, 4, 5, 6, 7, 8, 9, 10 or more samples.

In some embodiments, systems provided herein may be configured to process RNA.

However, in some embodiments, the system may be configured to process DNA. In some embodiments, different nucleic acids may be processed in series or in parallel within the system. In some embodiments, cartridges may be used to perform gene fusion assays in an automated fashion, for example, to detect genetic alterations in ALK, RET, or ROS1. Such assays are disclosed herein as well as in US Patent Application Publication Number US 2013/0303461, which was published on Nov. 14, 2013, US Patent Application Publication Number and US 2015/02011050, which was published on Jul. 20, 2013, the contents of each of which are incorporated herein by reference in their entirety. In some embodiments, systems provided herein can process in an automated fashion an Xgen protocol from Integrated DNA Technologies or other similar nucleic acid processing protocol.

In some embodiments, cartridge and cassettes will have all of the reagents needed for carrying out a particular protocol. In some embodiments, once a carrier is loaded into a cartridge bay an access door to that bay is closed, and optionally a lid (e.g., a heated lid) may be lowered automatically. In some embodiments, lowering of the lid (e.g., the heated lid) forces (or places) the cartridge down onto an array of heater jackets which conform to each of a set of one or more temperature controlled vessels in the cartridge. In some embodiments, this places the cartridge in a known position vertically in the drawer assembly. In some embodiments, lowering of the lid forces the cartridge down into a position in which rotary valves present in the cartridge are capable of engaging with corresponding drivers that control the rotational position of the valves in the cartridge. In some embodiments, automation components are provided to ensure that the rotary valves properly engage with their drivers.

In some embodiments of methods provided herein, a nucleic acid sample present in a cartridge (e.g., within a vessel of a cassette) will be mixed with a lyosphere. In some embodiments, the lyosphere will contain a fluorophore which will attach to the sample. In some embodiments, there will also be a “reference material” in the lyosphere which will contain a known amount of a molecule (e.g., of synthetic DNA). In some embodiments, attached to the “reference material” will be another fluorophore which will emit light at a different wavelength than the sample's fluorophore. In some embodiments, fluorophores used may be attached to the sample or the “reference material” via an intercalating dye (e.g., SYBR Green) or a reporter/quencher chemistry (e.g., TaqMan, etc.). In some embodiments, during quantitative PCR (qPCR) cycling the fluorescence of the two fluorophores will be monitored and then used to determine the amount of nucleic acid (e.g., DNA, cDNA) in the sample by the Comparative CT method.

Advantageously, certain systems described herein may include modular components (e.g., cassettes) that can allow tailoring of specific reactions and/or steps to be performed. In some embodiments, certain cassettes for performing a particular type of reaction are included in the cartridge. For example, cassettes including vessels containing lyospheres with different reagents for performing multiple steps of a PCR reaction may be present in the cartridge. The frame or cartridge may further include empty regions for a user to insert one or more cassettes containing specific fluids and/or reagents for a specific reaction (or set of reactions) to be performed in the cartridge. For example, a user may insert one or more cassettes containing particular buffers, reagents, alcohols, and/or primers into the frame or cartridge. Alternatively, a user may insert a different set of cassettes including a different set of fluids and/or reagents into the empty regions of the frame or cassette for performing a different reaction and/or experiment. After the cassettes are inserted into the frame or cartridge, they may form a fluidic connection with a channel system for transporting fluids to conduct the reactions/analyses.

In some embodiments, multiple analyses may be performed simultaneously or sequentially by inserting different cassettes into the cartridge. For instance, the systems and methods described herein may advantageously provide the ability to analyze two or more samples without the need to open the system or change the cartridge. For example, in some cases, one or more reactions with one or more samples may be conducted in parallel (e.g., conducting two or more PCR reactions in parallel). Such modularity and flexibility may allow for the analysis of multiple samples, each of which may require one or several reaction steps within a single fluidic system. Accordingly, multiple complex reactions and analyses may be performed using the systems and methods described herein.

Unlike certain existing fluidic systems and methods, the systems and methods described herein may be reusable (e.g., a reusable carrier plate) or disposable (e.g., consumable components including cassettes and various fluidic components). In some cases, the systems described herein may occupy a relatively small footprint as compared to certain existing fluidic systems for performing similar reactions and experiments.

In some embodiments, the cassettes and/or cartridge includes stored fluids and/or reagents needed to perform a particular reaction or analysis (or set of reactions or analyses) with one or more samples. Examples of cassettes include, but are not limited to, reagent cassettes, primer cassettes, buffer cassettes, waste cassettes, sample cassettes, and output cassettes. Other appropriate modules or cassettes may be used. Such cassettes may be configured in a manner that prevents or eliminates contamination or loss of the stored reagents prior to the use of those reagents. Other advantages are described in more detail below.

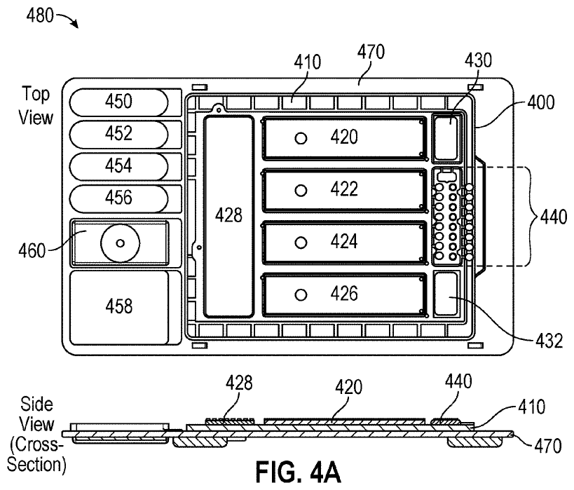

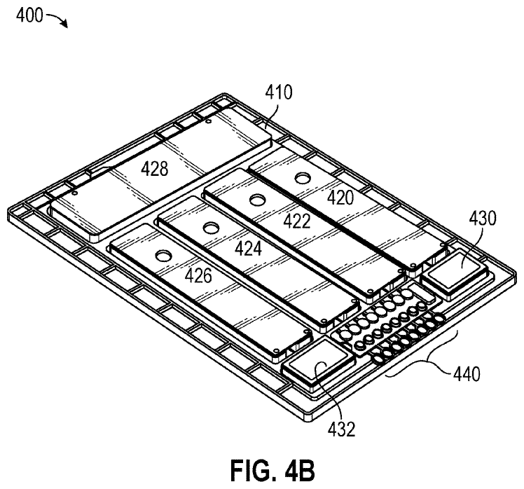

In one embodiment, as shown illustratively in FIGS. 4A and 4B, cartridge 400 comprises a frame 410 and cassettes 420, 422, 424, 426, 428, 430, 432, and 440. In some embodiments, each of these cassettes may be in fluidic communication with a channel system (e.g., positioned underneath the cassettes, not shown). In some embodiments, at least one of cassettes 428 (e.g., a reagent cassettes), 430 (e.g., a reagent cassette), and 432 (e.g., a reagent cassette) may be inserted into frame 410 by the user such that the cassettes are in fluidic communication with the channel system. For example, in some embodiments, one of cassettes 428, 430, and 432 is a reagent cassette containing a reaction buffer (e.g., Tris buffer). In certain embodiments, cassettes 428, 430 and/or 432 may comprise one or more reagents and/or reaction vessels for a reaction or a set of reactions. In some embodiments, module 440 comprises a plurality of sample wells and/or output wells (e.g., samples wells configured to receive one or more samples). In some cases, cassettes 420, 422, 424, and 426 may comprise one or more stored reagents or reactants (e.g., lyospheres). For instance, each of cassettes 420, 422, 424, and 426 may include different sets of stored reagents or reactants for performing separate reactions. For example, cassette 420 may include a first set of reagents for performing a first PCR reaction, and cassette 422 may include a second set of reagents for performing a second PCR reaction. The first and second reactions may be performed simultaneously (e.g., in parallel) or sequentially.

In some embodiments, as shown illustratively in FIG. 4A, a carrier plate assembly 480 comprises a carrier plate 470 and additional cassettes including modules 450, 452, 454, 456, 458, and 460. In an exemplary embodiment, cassettes 450, 452, 454, 456, 458, and 460 may each comprise one or more stored reagents and/or may be configured and arranged to receive one or more fluids (e.g., module 458 may be a waste module configured to collect reaction waste fluids). In some embodiments, one or more of cassettes 450, 452, 454, 456, 458, and 460 may be refillable.

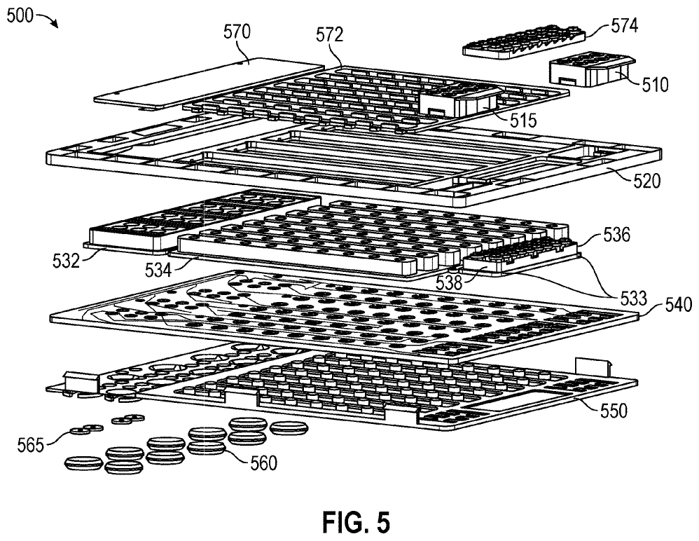

FIG. 5 is an exploded view of an exemplary cartridge 500, according to one set of embodiments. Cartridge 500 comprises a primer cassette 510 and a primer cassette 515 which may be inserted into one or more openings in a frame 520. Cartridge 500 further comprises a fluidics layer assembly 540 containing a channel system adjacent and non-integral to frame 520. In some embodiments, a set of cassettes 532 (e.g., comprising one or more primer cassettes, buffer cassettes, reagent cassettes, and/or waste cassettes, each optionally including one or more vessels), set of reaction cassettes 534, which comprises reaction vessels, an input/output cassette 533, which comprises sample input vessels 536 and output vessels 538, may be inserted into one or more openings in frame 520. In some embodiments, cartridge 500 comprises a valve plate 550. In some embodiments, valve plate 550 connects (e.g., snaps) into frame 520 and holds in place fluidics layer assembly 540 and cassettes 532, 533 and 534 in frame 520. In certain embodiments, cartridge 500 comprises valves 560, as described herein, and a plurality of seals 565. In some cases, frame 520 and/or one or more modules may be covered by covers 570, 572, and/or 574.

In some embodiments, a magnetic assembly can be used to separate one or more sample components or one or more reaction components from a liquid buffer and/or from other sample or reaction components. For example, in some embodiments magnetic particles that bind (e.g., selectively or specifically) to a molecule of interest (e.g., a nucleic acid or a specific target nucleic acid) may be used to isolate the molecule from a biological sample. In some embodiments, magnetic particles may be used to purify reaction products (e.g., nucleic acid amplification products) for further analysis (e.g., sequencing). Accordingly, magnetic assemblies and methods described in this application may be used to isolate or purify (e.g., partially or completely) one or more templates, intermediates, or reaction products described in the following processes.

### Amplification (AMP) Methods

Described herein are methods of determining the nucleotide sequence contiguous to a known target nucleotide sequence. The methods may be implemented in an automated fashion using the systems disclosed herein. Traditional sequencing methods generate sequence information randomly (e.g., “shotgun” sequencing) or between two known sequences which are used to design primers. In contrast, certain of the methods described herein, in some embodiments, allow for determining the nucleotide sequence (e.g., sequencing) upstream or downstream of a single region of known sequence with a high level of specificity and sensitivity.

In some embodiments, the systems provided herein may be configured to implement, e.g., in an automated fashion, a method of enriching specific nucleotide sequences prior to determining the nucleotide sequence using a next-generation sequencing technology. In some embodiments, methods provided herein can relate to enriching samples comprising deoxyribonucleic acid (DNA). In some embodiments, methods provided herein comprise: (a) ligating a target nucleic acid comprising the known target nucleotide sequence with a universal oligonucleotide tail-adapter; (b) amplifying a portion of the target nucleic acid and the amplification strand of the universal oligonucleotide tail-adapter with a first adapter primer and a first target-specific primer; (c) amplifying a portion of the amplicon resulting from step (b) with a second adapter primer and a second target-specific primer; and (d) transferring the DNA solution to a user. In some embodiments, one or more steps of the methods may be performed within different vessels of a cartridge provided herein. In some embodiments, microfluidic channels and valves in the cartridge facilitate the transfer of reaction material/fluid from one vessel to another in the cartridge to permit reactions to proceed in an automated fashion. In some embodiments, a DNA solution can subsequently be sequenced with a first and second sequencing primer using a next-generation sequencing technology.

In some embodiments, a sample processed using a system provided herein comprises genomic DNA. In some embodiments, samples comprising genomic DNA include a fragmentation step preceding step (a). In some embodiments, each ligation and amplification step can optionally comprise a subsequent purification step (e.g., sample purification between step (a) and step (b), sample purification between step (b) and step (c), and/or sample purification following step (c)). For example, the method of enriching samples comprising genomic DNA can comprise: (a) fragmentation of genomic DNA; (b) ligating a target nucleic acid comprising the known target nucleotide sequence with a universal oligonucleotide tail-adapter; (c) post-ligation sample purification; (d) amplifying a portion of the target nucleic acid and the amplification strand of the universal oligonucleotide tail-adapter with a first adapter primer and a first target-specific primer; (e) post-amplification sample purification; (f) amplifying a portion of the amplicon resulting from step (d) with a second adapter primer and a second target-specific primer; (g) post-amplification sample purification; and (h) transferring the purified DNA solution to a user. In some embodiments, steps of the methods may be performed within different vessels of a cartridge provided herein. In some embodiments, microfluidic channels and valves in the cartridge facilitate the transfer of reaction material/fluid from one vessel to another in the cartridge in an automated fashion. In The purified sample can subsequently be sequenced with a first and second sequencing primer using a next-generation sequencing technology.

In some embodiments, systems and methods provided herein may be used for processing nucleic acids as depicted in the exemplary workflow in FIG. 1. A nucleic acid sample 120 is provided. In some embodiments, the sample comprises RNA. In some embodiments, the sample comprises DNA (e.g., double-stranded complementary DNA (cDNA) and/or double-stranded genomic DNA (gDNA) 102). In some embodiments, the nucleic acid sample is subjected to a step 102 comprising nucleic acid end repair and/or dA tailing. In some embodiments, the nucleic acid sample is subjected to a step 104 comprising adapter ligation. In some embodiments, a universal oligonucleotide adapter 122 is ligated to one or more nucleic acids in the nucleic acid sample. In some embodiments, the ligation step comprises blunt-end ligation. In some embodiments, the ligation step comprises sticky-end ligation. In some embodiments, the ligation step comprises overhang ligation. In some embodiments, the ligation step comprises TA ligation. In some embodiments, the dA tailing step 102 is performed to generate an overhang in the nucleic acid sample that is complementary to an overhang in the universal oligonucleotide adapter (e.g., TA ligation). In some embodiments, a universal oligonucleotide adapter is ligated to both ends of one or more nucleic acids in the nucleic acid sample to generate a nucleic acid 124 flanked by universal oligonucleotide adapters. In some embodiments, an initial round of amplification is performed using an adapter primer 130 and a first target-specific primer 132. In some embodiments, the amplified sample is subjected to a second round of amplification using an adapter primer and a second target-specific primer 134. In some embodiments, the second target-specific primer is nested relative to the first target-specific primer. In some embodiments, the second target-specific primer comprises additional sequences 5′ to a hybridization sequence (e.g., common sequence) that may include barcode, index, adapter sequences, or sequencing primer sites. In some embodiments, the second target-specific primer is further contacted by an additional primer that hybridizes with the common sequence of the second target-specific primer, as depicted by 134. In some embodiments, the second round of amplification generates a nucleic acid 126 that is suitable for nucleic acid sequencing (e.g., next generation sequencing methods). In some embodiments, systems and methods provided herein may be used for processing nucleic acids as described in PCT International Application No. PCT/US2017/051924, which was filed on Sep. 15, 2017, and which claims priority under 35 U.S.C. § 119(e) to U.S. Provisional Patent Application No. 62/395,339, which was filed on Sep. 15, 2016, and in PCT International Application No. PCT/US2017/051927, which was filed on Sep. 15, 2017, and which claims priority under 35 U.S.C. § 119(e) to U.S. Provisional Patent Application No. 62/395,347, which was filed on Sep. 15, 2016, the entire contents of each of which relating to nucleic acid library preparation are hereby incorporated by reference.

In some embodiments, a sample processed using a system provided herein comprises ribonucleic acid (RNA). In some embodiments, a system provided herein can be useful for processing RNA by a method comprising: (a) contacting a target nucleic acid molecule comprising the known target nucleotide sequence with a population of random primers under hybridization conditions; (b) performing a template-dependent extension reaction that is primed by a hybridized random primer and that uses the portion of the target nucleic acid molecule downstream of the site of hybridization as a template; (c) contacting the product of step (b) with an initial target-specific primer under hybridization conditions; (d) performing a template-dependent extension reaction that is primed by a hybridized initial target-specific primer and that uses the target nucleic acid molecule as a template; (e) subjecting the nucleic acid to end-repair, phosphorylation, and adenylation; (f) ligating the target nucleic acid comprising the known target nucleotide sequence with a universal oligonucleotide tail-adapter; (g) amplifying a portion of the target nucleic acid and the amplification strand of the universal oligonucleotide tail-adapter with a first adapter primer and a first target-specific primer; (h) amplifying a portion of the amplicon resulting from step (g) with a second adapter primer and a second target-specific primer; and (i) transferring the cDNA solution to a user. In some embodiments, one or more steps of the methods may be performed within different vessels of a cartridge provided herein. In some embodiments, cDNA solution can subsequently be sequenced with a first and second sequencing primer using a next-generation sequencing technology.

In some embodiments, each ligation and amplification step can optionally comprise a subsequent sample purification step (e.g., sample purification step between step (f) and step (g), sample purification step between step (g) and step (h), and/or sample purification following step (h)). For example, the method of enriching samples comprising RNA can comprise: (a) contacting a target nucleic acid molecule comprising the known target nucleotide sequence with a population of random primers under hybridization conditions; (b) performing a template-dependent extension reaction that is primed by a hybridized random primer and that uses the portion of the target nucleic acid molecule downstream of the site of hybridization as a template; (c) contacting the product of step (b) with an initial target-specific primer under hybridization conditions; (d) performing a template-dependent extension reaction that is primed by a hybridized initial target-specific primer and that uses the target nucleic acid molecule as a template; (e) subjecting the nucleic acid to end-repair, phosphorylation, and adenylation; (f) ligating the target nucleic acid comprising the known target nucleotide sequence with a universal oligonucleotide tail-adapter; (g) post-ligation sample purification; (h) amplifying a portion of the target nucleic acid and the amplification strand of the universal oligonucleotide tail-adapter with a first adapter primer and a first target-specific primer; (i) post-amplification sample purification; (j) amplifying a portion of the amplicon resulting from step (h) with a second adapter primer and a second target-specific primer; (k) post-amplification sample purification; and (l) transferring the purified cDNA solution to a user. In some embodiments, one or more steps of the methods may be performed within different vessels of a cartridge provided herein. The purified sample can subsequently be sequenced with a first and second sequencing primer using a next-generation sequencing technology.

In some embodiments, the systems provided herein may be configured to implement, e.g., in an automated fashion, a method of enriching nucleotide sequences that comprise a known target nucleotide sequence downstream from an adjacent region of unknown nucleotide sequence (e.g., nucleotide sequences comprising a 5′ region comprising an unknown sequence and a 3′ region comprising a known sequence). In some embodiments, the method comprises: (a) contacting a target nucleic acid molecule comprising the known target nucleotide sequence with an initial target-specific primer under hybridization conditions; (b) performing a template-dependent extension reaction that is primed by a hybridized initial target-specific primer and that uses the target nucleic acid molecule as a template; (c) contacting the product of step (b) with a population of tailed random primers under hybridization conditions; (d) performing a template-dependent extension reaction that is primed by a hybridized tailed random primer and that uses the portion of the target nucleic acid molecule downstream of the site of hybridization as a template; (e) amplifying a portion of the target nucleic acid molecule and the tailed random primer sequence with a first tail primer and a first target-specific primer; (f) amplifying a portion of the amplicon resulting from step (e) with a second tail primer and a second target-specific primer; and (g) transferring the cDNA solution to a user. The cDNA solution can subsequently be sequenced with a first and second sequencing primer using a next-generation sequencing technology. In some embodiments, the population of tailed random primers comprises single-stranded oligonucleotide molecules having a 5′ nucleic acid sequence identical to a first sequencing primer and a 3′ nucleic acid sequence comprising from about 6 to about 12 random nucleotides. In some embodiments, the first target-specific primer comprises a nucleic acid sequence that can specifically anneal to the known target nucleotide sequence of the target nucleic acid at the annealing temperature. In some embodiments, the second target-specific primer comprises a 3′ portion comprising a nucleic acid sequence that can specifically anneal to a portion of the known target nucleotide sequence comprised by the amplicon resulting from step (e), and a 5′ portion comprising a nucleic acid sequence that is identical to a second sequencing primer and the second target-specific primer is nested with respect to the first target-specific primer. In some embodiments, the first tail primer comprises a nucleic acid sequence identical to the tailed random primer. In some embodiments, the second tail primer comprises a nucleic acid sequence identical to a portion of the first sequencing primer and is nested with respect to the first tail primer. In some embodiments, one or more steps of the method may be performed within different vessels of a cartridge provided herein. In some embodiments, the systems provided herein may be configured to implement, e.g., in an automated fashion, a method of enriching nucleotide sequences that comprise a known target nucleotide sequence upstream from an adjacent region of unknown nucleotide sequence (e.g., nucleotide sequences comprising a 5′ region comprising a known sequence and a 3′ region comprising an unknown sequence). In some embodiments, the method comprises: (a) contacting a target nucleic acid molecule comprising the known target nucleotide sequence with a population of tailed random primers under hybridization conditions; (b) performing a template-dependent extension reaction that is primed by a hybridized tailed random primer and that uses the portion of the target nucleic acid molecule downstream of the site of hybridization as a template; (c) contacting the product of step (b) with an initial target-specific primer under hybridization conditions; (d) performing a template-dependent extension reaction that is primed by a hybridized initial target-specific primer and that uses the target nucleic acid molecule as a template; (e) amplifying a portion of the target nucleic acid molecule and the tailed random primer sequence with a first tail primer and a first target-specific primer; (f) amplifying a portion of the amplicon resulting from step (e) with a second tail primer and a second target-specific primer; and (g) transferring the cDNA solution to a user. The cDNA solution can subsequently be sequenced with a first and second sequencing primer using a next-generation sequencing technology. In some embodiments, the population of tailed random primers comprises single-stranded oligonucleotide molecules having a 5′ nucleic acid sequence identical to a first sequencing primer and a 3′ nucleic acid sequence comprising from about 6 to about 12 random nucleotides. In some embodiments, the first target-specific primer comprises a nucleic acid sequence that can specifically anneal to the known target nucleotide sequence of the target nucleic acid at the annealing temperature. In some embodiments, the second target-specific primer comprises a 3′ portion comprising a nucleic acid sequence that can specifically anneal to a portion of the known target nucleotide sequence comprised by the amplicon resulting from step (c), and a 5′ portion comprising a nucleic acid sequence that is identical to a second sequencing primer and the second target-specific primer is nested with respect to the first target-specific primer. In some embodiments, the first tail primer comprises a nucleic acid sequence identical to the tailed random primer. In some embodiments, the second tail primer comprises a nucleic acid sequence identical to a portion of the first sequencing primer and is nested with respect to the first tail primer. In some embodiments, one or more steps of the method may be performed within different vessels of a cartridge provided herein. In some embodiments, the method further involves a step of contacting the sample with RNase after extension of the initial target-specific primer. In some embodiments, the tailed random primer can form a hair-pin loop structure. In some embodiments, the initial target-specific primer and the first target-specific primer are identical. In some embodiments, the tailed random primer further comprises a barcode portion comprising 6-12 random nucleotides between the 5′ nucleic acid sequence identical to a first sequencing primer and the 3′ nucleic acid sequence comprising 6-12 random nucleotides.

### Universal Oligonucleotide Tail Adapter

As used herein, the term “universal oligonucleotide tail-adapter” refers to a nucleic acid molecule comprised of two strands (a blocking strand and an amplification strand) and comprising a first ligatable duplex end and a second unpaired end. The blocking strand of the universal oligonucleotide tail-adapter comprises a 5′ duplex portion. The amplification strand comprises an unpaired 5′ portion, a 3′ duplex portion, a 3′ T overhang, and nucleic acid sequences identical to a first and second sequencing primer. The duplex portions of the blocking strand and the amplification strand are substantially complementary and form the first ligatable duplex end comprising a 3′ T overhang and the duplex portion is of sufficient length to remain in duplex form at the ligation temperature.

In some embodiments, the portion of the amplification strand that comprises a nucleic acid sequence identical to a first and second sequencing primer can be comprised, at least in part, by the 5′ unpaired portion of the amplification strand.

In some embodiments, the universal oligonucleotide tail-adapter can comprise a duplex portion and an unpaired portion, wherein the unpaired portion comprises only the 5′ portion of the amplification strand, i.e., the entirety of the blocking strand is a duplex portion.

In some embodiments, the universal oligonucleotide tail-adapter can have a “Y” shape, i.e., the unpaired portion can comprise portions of both the blocking strand and the amplification strand which are unpaired. The unpaired portion of the blocking strand can be shorter than, longer than, or equal in length to the unpaired portion of the amplification strand. In some embodiments, the unpaired portion of the blocking strand can be shorter than the unpaired portion of the amplification strand. Y shaped universal oligonucleotide tail-adapters have the advantage that the unpaired portion of the blocking strand will not be subject to 3′ extension during a PCR regimen.

In some embodiments, the blocking strand of the universal oligonucleotide tail-adapter can further comprise a 3′ unpaired portion which is not substantially complementary to the 5′ unpaired portion of the amplification strand; and wherein the 3′ unpaired portion of the blocking strand is not substantially complementary to or substantially identical to any of the primers. In some embodiments, the blocking strand of the universal oligonucleotide tail-adapter can further comprise a 3′ unpaired portion which will not specifically anneal to the 5′ unpaired portion of the amplification strand at the annealing temperature; and wherein the 3′ unpaired portion of the blocking strand will not specifically anneal to any of the primers or the complements thereof at the annealing temperature.

### First Amplification Step

As used herein, the term “first target-specific primer” refers to a single-stranded oligonucleotide comprising a nucleic acid sequence that can specifically anneal under suitable annealing conditions to a nucleic acid template that has a strand characteristic of a target nucleic acid.

In some embodiments, a primer (e.g., a target specific primer) can comprise a 5′ tag sequence portion. In some embodiments, multiple primers (e.g., all first-target specific primers) present in a reaction can comprise identical 5′ tag sequence portions. In some embodiments, in a multiplex PCR reaction, different primer species can interact with each other in an off-target manner, leading to primer extension and subsequently amplification by DNA polymerase. In such embodiments, these primer dimers tend to be short, and their efficient amplification can overtake the reaction and dominate resulting in poor amplification of desired target sequence. Accordingly, in some embodiments, the inclusion of a 5′ tag sequence in primers (e.g., on target specific primer(s)) may result in formation of primer dimers that contain the same complementary tails on both ends. In some embodiments, in subsequent amplification cycles, such primer dimers would denature into single-stranded DNA primer dimers, each comprising complementary sequences on their two ends which are introduced by the 5′ tag. In some embodiments, instead of primer annealing to these single stranded DNA primer dimers, an intra-molecular hairpin (a panhandle like structure) formation may occur due to the proximate accessibility of the complementary tags on the same primer dimer molecule instead of an inter-molecular interaction with new primers on separate molecules. Accordingly, in some embodiments, these primer dimers may be inefficiently amplified, such that primers are not exponentially consumed by the dimers for amplification; rather the tagged primers can remain in high and sufficient concentration for desired specific amplification of target sequences. In some embodiments, accumulation of primer dimers may be undesirable in the context of multiplex amplification because they compete for and consume other reagents in the reaction.

In some embodiments, a 5′ tag sequence can be a GC-rich sequence. In some embodiments, a 5′ tag sequence may comprise at least 50% GC content, at least 55% GC content, at least 60% GC content, at least 65% GC content, at least 70% GC content, at least 75% GC content, at least 80% GC content, or higher GC content. In some embodiments, a tag sequence may comprise at least 60% GC content. In some embodiments, a tag sequence may comprise at least 65% GC content.

As used herein, the term “first adapter primer” refers to a nucleic acid molecule comprising a nucleic acid sequence identical to a 5′ portion of the first sequencing primer. As the first tail-adapter primer is therefore identical to at least a portion of the sequence of the amplification strand (as opposed to complementary), it will not be able to specifically anneal to any portion of the universal oligonucleotide tail-adapter itself.

In the first PCR amplification cycle of the first amplification step, the first target-specific primer can specifically anneal to a template strand of any nucleic acid comprising the known target nucleotide sequence. Depending upon the orientation with which the first target-specific primer was designed, a sequence upstream or downstream of the known target nucleotide sequence will be synthesized as a strand complementary to the template strand. If, during the extension phase of PCR, the 5′ end of the template strand terminates in a ligated universal oligonucleotide tail-adapter, the 3′ end of the newly synthesized product strand will comprise sequence complementary to the first tail-adapter primer. In subsequent PCR amplification cycles, both the first target-specific primer and the first tail-adapter primer will be able to specifically anneal to the appropriate strands of the target nucleic acid sequence and the sequence between the known nucleotide target sequence and the universal oligonucleotide tail-adapter can be amplified (i.e., copied).

### Second Amplification Step

As used herein, the term “second target-specific primer” refers to a single-stranded oligonucleotide comprising a 3′ portion comprising a nucleic acid sequence that can specifically anneal to a portion of the known target nucleotide sequence comprised by the amplicon resulting from a preceding amplification step, and a 5′ portion comprising a nucleic acid sequence that is identical to a second sequencing primer. The second target-specific primer can be further contacted by an additional primer (e.g., a primer having 3′ sequencing adapter/index sequences) that hybridizes with the common sequence of the second target-specific primer. In some embodiments, the additional primer may comprise additional sequences 5′ to the hybridization sequence that may include barcode, index, adapter sequences, or sequencing primer sites. In some embodiments, the additional primer is a generic sequencing adapter/index primer. The second target-specific primer is nested with respect to the first target-specific primer. In some embodiments, the second target-specific primer is nested with respect to the first target-specific primer by at least 3 nucleotides, e.g., by 3 or more, 4 or more, 5 or more, 6 or more, 7 or more, 8 or more, 9 or more, 10 or more, or 15 or more nucleotides.

In some embodiments, all of the second target-specific primers present in a reaction comprise the same 5′ portion. In some embodiments, the 5′ portion of the second target-specific primers can serve to suppress primer dimers as described for the 5′ tag of the first target-specific primer described above herein.

In some embodiments, the first and second target-specific primers are substantially complementary to the same strand of the target nucleic acid. In some embodiments, the portions of the first and second target-specific primers that specifically anneal to the known target sequence can comprise a total of at least 20 unique bases of the known target nucleotide sequence, e.g., 20 or more unique bases, 25 or more unique bases, 30 or more unique bases, 35 or more unique bases, 40 or more unique bases, or 50 or more unique bases. In some embodiments, the portions of the first and second target-specific primers that specifically anneal to the known target sequence can comprise a total of at least 30 unique bases of the known target nucleotide sequence.

As used herein, the term “second adapter primer” refers to a nucleic acid molecule comprising a nucleic acid sequence identical to a portion of the first sequencing primer and is nested with respect to the first adapter primer. As the second tail-adapter primer is therefore identical to at least a portion of the sequence of the amplification strand (as opposed to complementary), it will not be able to specifically anneal to any portion of the universal oligonucleotide tail-adapter itself. In some embodiments, the second adapter primer is identical to the first sequencing primer.

The second adapter primer should be nested with respect to the first adapter primer, that is, the first adapter primer comprises a nucleic acid sequence identical to the amplification strand which is not comprised by the second adapter primer and which is located closer to the 5′ end of the amplification primer than any of the sequence identical to the amplification strand which is comprised by the second adapter primer. In some embodiments, the second adapter primer is nested by at least 3 nucleotides, e.g., by 3 nucleotides, by 4 nucleotides, by 5 nucleotides, by 6 nucleotides, by 7 nucleotides, by 8 nucleotides, by 9 nucleotides, by 10 nucleotides or more.

In some embodiments, the first adapter primer can comprise a nucleic acid sequence identical to about the 20 5′-most bases of the amplification strand of the universal oligonucleotide tail-adapter and the second adapter primer can comprise a nucleic acid sequence identical to about 30 bases of the amplification strand of the universal oligonucleotide tail-adapter, with a 5′ base which is at least 3 nucleotides 3′ of the 5′ terminus of the amplification strand.

In some embodiments, nested primer sets may be used. In some embodiments, the use of nested adapter primers eliminates the possibility of producing final amplicons that are amplifiable (e.g., during bridge PCR or emulsion PCR) but cannot be efficiently sequenced using certain techniques. In some embodiments, hemi-nested primer sets may be used.

### Sample Purification Step

In some embodiments, target nucleic acids and/or amplification products thereof can be isolated from enzymes, primers, or buffer components before and/or after any appropriate step of a method. Any suitable methods for isolating nucleic acids may be used. In some embodiments, the isolation can comprise Solid Phase Reversible Immobilization (SPRI) cleanup. Methods for SPRI cleanup are well known in the art, e.g., Agencourt AMPure XP—PCR Purification (Cat No. A63880, Beckman Coulter; Brea, Calif.). In some embodiments, enzymes can be inactivated by heat treatment.

In some embodiments, unhybridized primers can be removed from a nucleic acid preparation using appropriate methods (e.g., purification, digestion, etc.). In some embodiments, a nuclease (e.g., exonuclease I) is used to remove primer from a preparation.

In some embodiments, such nucleases are heat inactivated subsequent to primer digestion. Once the nucleases are inactivated, a further set of primers may be added together with other appropriate components (e.g., enzymes, buffers) to perform a further amplification reaction.

In some embodiments, unhybridized primers, buffers, salts, enzymes, etc., or any combination thereof can be removed from a nucleic acid preparation using magnetic particles that bind to the nucleic acid of interest and a magnetic assembly as described in this application.

### Sequencing

In some aspects, the technology described herein relates to methods of enriching nucleic acid samples for oligonucleotide sequencing. In some embodiments, the sequencing can be performed by a next-generation sequencing method. As used herein, “next-generation sequencing” refers to oligonucleotide sequencing technologies that have the capacity to sequence oligonucleotides at speeds above those possible with conventional sequencing methods (e.g., Sanger sequencing), due to performing and reading out thousands to millions of sequencing reactions in parallel. Non-limiting examples of next-generation sequencing methods/platforms include Massively Parallel Signature Sequencing (Lynx Therapeutics); 454 pyro-sequencing (454 Life Sciences/Roche Diagnostics); solid-phase, reversible dye-terminator sequencing (Solexa/Illumina); SOLiD technology (Applied Biosystems); Ion semiconductor sequencing (ION Torrent); DNA nanoball sequencing (Complete Genomics); and technologies available from Pacific Biosciences, Intelligen Bio-systems, and Oxford Nanopore Technologies. In some embodiments, the sequencing primers can comprise portions compatible with the selected next-generation sequencing method. Next-generation sequencing technologies and the constraints and design parameters of associated sequencing primers are well known in the art (see, e.g., Shendure, et al., “Next-generation DNA sequencing,” Nature, 2008, vol. 26, No. 10, 1135-1145; Mardis, “The impact of next-generation sequencing technology on genetics,” Trends in Genetics, 2007, vol. 24, No. 3, pp. 133-141; Su, et al., “Next-generation sequencing and its applications in molecular diagnostics” Expert Rev Mol Diagn, 2011, 11(3):333-43; Zhang et al., “The impact of next-generation sequencing on genomics”, J Genet Genomics, 2011, 38(3):95-109; (Nyren, P. et al. Anal Biochem 208: 17175 (1993); Bentley, D. R. Curr Opin Genet Dev 16:545-52 (2006); Strausberg, R. L., et al. Drug Disc Today 13:569-77 (2008); U.S. Pat. Nos. 7,282,337; 7,279,563; 7,226,720; 7,220,549; 7,169,560; 6,818,395; 6,911,345; US Pub. Nos. 2006/0252077; 2007/0070349; and 20070070349; which are incorporated by reference herein in their entireties).

In some embodiments, the sequencing step relies upon the use of a first and second sequencing primer. In some embodiments, the first and second sequencing primers are selected to be compatible with a next-generation sequencing method as described herein.

Methods of aligning sequencing reads to known sequence databases of genomic and/or cDNA sequences are well known in the art, and software is commercially available for this process. In some embodiments, reads (less the sequencing primer and/or adapter nucleotide sequence) which do not map, in their entirety, to wild-type sequence databases can be genomic rearrangements or large indel mutations. In some embodiments, reads (less the sequencing primer and/or adapter nucleotide sequence) comprising sequences which map to multiple locations in the genome can be genomic rearrangements.

### AMP Primers

In some embodiments, the four types of primers (first and second target-specific primers and first and second adapter primers) are designed such that they will specifically anneal to their complementary sequences at an annealing temperature of from about 61 to 72° C., e.g., from about 61 to 69° C., from about 63 to 69° C., from about 63 to 67° C., from about 64 to 66° C. In some embodiments, the four types of primers are designed such that they will specifically anneal to their complementary sequences at an annealing temperature of less than 72° C. In some embodiments, the four types of primers are designed such that they will specifically anneal to their complementary sequences at an annealing temperature of less than 70° C. In some embodiments, the four types of primers are designed such that they will specifically anneal to their complementary sequences at an annealing temperature of less than 68° C. In some embodiments, the four types of primers are designed such that they will specifically anneal to their complementary sequences at an annealing temperature of about 65° C. In some embodiments, systems provided herein are configured to alter vessel temperature (e.g., by cycling between different temperature ranges) to facilitate primer annealing.

In some embodiments, the portions of the target-specific primers that specifically anneal to the known target nucleotide sequence will anneal specifically at a temperature of about 61 to 72° C., e.g., from about 61 to 69° C., from about 63 to 69° C., from about 63 to 67° C., from about 64 to 66° C. In some embodiments, the portions of the target-specific primers that specifically anneal to the known target nucleotide sequence will anneal specifically at a temperature of about 65° C. in a PCR buffer.

In some embodiments, the primers and/or adapters described herein cannot comprise modified bases (e.g., the primers and/or adapters cannot comprise a blocking 3′ amine).

### Nucleic Acid Extension, Amplification, and PCR

In some embodiments, methods described herein comprise an extension regimen or step. In such embodiments, extension may proceed from one or more hybridized tailed random primers, using the nucleic acid molecules which the primers are hybridized to as templates. Extension steps are described herein. In some embodiments, one or more tailed random primers can hybridize to substantially all of the nucleic acids in a sample, many of which may not comprise a known target nucleotide sequence. Accordingly, in some embodiments, extension of random primers may occur due to hybridization with templates that do not comprise a known target nucleotide sequence.

In some embodiments, methods described herein may involve a polymerase chain reaction (PCR) amplification regimen, involving one or more amplification cycles. Amplification steps of the methods described herein can each comprise a PCR amplification regimen, i.e., a set of polymerase chain reaction (PCR) amplification cycles. In some embodiments, systems provided herein are configured to alter vessel temperature (e.g., by cycling between different temperature ranges) to facilitate different PCR steps, e.g., melting, annealing, elongation, etc.

In some embodiments, system provided herein are configured to implement an amplification regimen in an automated fashion. As used herein, the term “amplification regimen” refers to a process of specifically amplifying (increasing the abundance of) a nucleic acid of interest. In some embodiments, exponential amplification occurs when products of a previous polymerase extension serve as templates for successive rounds of extension. In some embodiments, a PCR amplification regimen according to methods disclosed herein may comprise at least one, and in some cases at least 5 or more iterative cycles. In some embodiments, each iterative cycle comprises steps of: 1) strand separation (e.g., thermal denaturation); 2) oligonucleotide primer annealing to template molecules; and 3) nucleic acid polymerase extension of the annealed primers. In should be appreciated that any suitable conditions and times involved in each of these steps may be used. In some embodiments, conditions and times selected may depend on the length, sequence content, melting temperature, secondary structural features, or other factors relating to the nucleic acid template and/or primers used in the reaction. In some embodiments, an amplification regimen according to methods described herein is performed in a thermal cycler, many of which are commercially available.

In some embodiments, a nucleic acid extension reaction involves the use of a nucleic acid polymerase. As used herein, the phrase “nucleic acid polymerase” refers an enzyme that catalyzes the template-dependent polymerization of nucleoside triphosphates to form primer extension products that are complementary to the template nucleic acid sequence. A nucleic acid polymerase enzyme initiates synthesis at the 3′ end of an annealed primer and proceeds in the direction toward the 5′ end of the template. Numerous nucleic acid polymerases are known in the art and are commercially available. One group of nucleic acid polymerases are thermostable, i.e., they retain function after being subjected to temperatures sufficient to denature annealed strands of complementary nucleic acids, e.g., 94° C., or sometimes higher. A non-limiting example of a protocol for amplification involves using a polymerase (e.g., Phoenix Taq, VeraSeq) under the following conditions: 98° C. for 30 s, followed by 14-22 cycles comprising melting at 98° C. for 10 s, followed by annealing at 68° C. for 30 s, followed by extension at 72° C. for 3 min, followed by holding of the reaction at 4° C. However, other appropriate reaction conditions may be used. In some embodiments, annealing/extension temperatures may be adjusted to account for differences in salt concentration (e.g., 3° C. higher to higher salt concentrations). In some embodiments, slowing the ramp rate (e.g., 1° C./s, 0.5° C./s, 0.28° C./s, 0.1° C./s or slower), for example, from 98° C. to 65° C., improves primer performance and coverage uniformity in highly multiplexed samples. In some embodiments, systems provided herein are configured to alter vessel temperature (e.g., by cycling between different temperature ranges, having controlled ramp up or down rates) to facilitate amplification.

In some embodiments, a nucleic acid polymerase is used under conditions in which the enzyme performs a template-dependent extension. In some embodiments, the nucleic acid polymerase is DNA polymerase I, Taq polymerase, Phoenix Taq polymerase, Phusion polymerase, T4 polymerase, T7 polymerase, Klenow fragment, Klenow exo-, phi29 polymerase, AMV reverse transcriptase, M-MuLV reverse transcriptase, HIV-1 reverse transcriptase, VeraSeq ULtra polymerase, VeraSeq HF 2.0 polymerase, EnzScript, or another appropriate polymerase. In some embodiments, a nucleic acid polymerase is not a reverse transcriptase. In some embodiments, a nucleic acid polymerase acts on a DNA template. In some embodiments, the nucleic acid polymerase acts on an RNA template. In some embodiments, an extension reaction involves reverse transcription performed on an RNA to produce a complementary DNA molecule (RNA-dependent DNA polymerase activity). In some embodiments, a reverse transcriptase is a mouse moloney murine leukemia virus (M-MLV) polymerase, AMV reverse transcriptase, RSV reverse transcriptase, HIV-1 reverse transcriptase, HIV-2 reverse transcriptase, or another appropriate reverse transcriptase.

In some embodiments, a nucleic acid amplification reaction involves cycles including a strand separation step generally involving heating of the reaction mixture. As used herein, the term “strand separation” or “separating the strands” means treatment of a nucleic acid sample such that complementary double-stranded molecules are separated into two single strands available for annealing to an oligonucleotide primer. In some embodiments, strand separation according to methods described herein is achieved by heating the nucleic acid sample above its melting temperature (Tₘ). In some embodiments, for a sample containing nucleic acid molecules in a reaction preparation suitable for a nucleic acid polymerase, heating to 94° C. is sufficient to achieve strand separation. In some embodiments, a suitable reaction preparation contains one or more salts (e.g., 1 to 100 mM KCl, 0.1 to 10 mM MgCl₂), at least one buffering agent (e.g., 1 to 20 mM Tris-HCl), and a carrier (e.g., 0.01 to 0.5% BSA). A non-limiting example of a suitable buffer comprises 50 mM KCl, 10 mM Tris-HCl (pH 8.8 at 25° C.), 0.5 to 3 mM MgCl₂, and 0.1% BSA.

In some embodiments, a nucleic acid amplification involves annealing primers to nucleic acid templates having a strands characteristic of a target nucleic acid. In some embodiments, a strand of a target nucleic acid can serve as a template nucleic acid.

As used herein, the term “anneal” refers to the formation of one or more complementary base pairs between two nucleic acids. In some embodiments, annealing involves two complementary or substantially complementary nucleic acid strands hybridizing together. In some embodiments, in the context of an extension reaction, annealing involves the hybridization of primer to a template such that a primer extension substrate for a template-dependent polymerase enzyme is formed. In some embodiments, conditions for annealing (e.g., between a primer and nucleic acid template) may vary based of the length and sequence of a primer. In some embodiments, conditions for annealing are based upon a Tₘ (e.g., a calculated Tₘ) of a primer. In some embodiments, an annealing step of an extension regimen involves reducing the temperature following a strand separation step to a temperature based on the Tₘ, (e.g., a calculated Tₘ) for a primer, for a time sufficient to permit such annealing. In some embodiments, a Tₘ can be determined using any of a number of algorithms (e.g., OLIGO™ (Molecular Biology Insights Inc. Colorado) primer design software and VENTRO NTI™ (Invitrogen, Inc. California) primer design software and programs available on the internet, including Primer3, Oligo Calculator, and NetPrimer (Premier Biosoft; Palo Alto, Calif.; and freely available on the world wide web (e.g., at premierbiosoft.com/netprimer/netprlaunch/Help/xnetprlaunch.html)). In some embodiments, the Tₘ of a primer can be calculated using the following formula, which is used by NetPrimer software and is described in more detail in Frieir, et al. PNAS 1986 83:9373-9377 which is incorporated by reference herein in its entirety.

Tₘ=ΔH/(ΔS+R*ln(C/4))+16.6 log([K⁺]/(1+0.7[K⁺]))−273.15

wherein: ΔH is enthalpy for helix formation; ΔS is entropy for helix formation; R is molar gas constant (1.987 cal/° C.*mol); C is the nucleic acid concentration; and [K⁺] is salt concentration. For most amplification regimens, the annealing temperature is selected to be about 5° C. below the predicted Tₘ, although temperatures closer to and above the Tₘ (e.g., between 1° C. and 5° C. below the predicted Tₘ or between 1° C. and 5° C. above the predicted Tₘ) can be used, as can, for example, temperatures more than 5° C. below the predicted Tₘ (e.g., 6° C. below, 8° C. below, 10° C. below or lower). In some embodiments, the closer an annealing temperature is to the Tₘ, the more specific is the annealing. In some embodiments, the time used for primer annealing during an extension reaction (e.g., within the context of a PCR amplification regimen) is determined based, at least in part, upon the volume of the reaction (e.g., with larger volumes involving longer times). In some embodiments, the time used for primer annealing during an extension reaction (e.g., within the context of a PCR amplification regimen) is determined based, at least in part, upon primer and template concentrations (e.g., with higher relative concentrations of primer to template involving less time than lower relative concentrations). In some embodiments, depending upon volume and relative primer/template concentration, primer annealing steps in an extension reaction (e.g., within the context of an amplification regimen) can be in the range of 1 second to 5 minutes, 10 seconds to 2 minutes, or 30 seconds to 2 minutes. As used herein, “substantially anneal” refers to an extent to which complementary base pairs form between two nucleic acids that, when used in the context of a PCR amplification regimen, is sufficient to produce a detectable level of a specifically amplified product.

As used herein, the term “polymerase extension” refers to template-dependent addition of at least one complementary nucleotide, by a nucleic acid polymerase, to the 3′ end of a primer that is annealed to a nucleic acid template. In some embodiments, polymerase extension adds more than one nucleotide, e.g., up to and including nucleotides corresponding to the full length of the template. In some embodiments, conditions for polymerase extension are based, at least in part, on the identity of the polymerase used. In some embodiments, the temperature used for polymerase extension is based upon the known activity properties of the enzyme. In some embodiments, in which annealing temperatures are below the optimal temperatures for the enzyme, it may be acceptable to use a lower extension temperature. In some embodiments, enzymes may retain at least partial activity below their optimal extension temperatures. In some embodiments, a polymerase extension (e.g., performed with thermostable polymerases such as Taq polymerase and variants thereof) is performed at 65° C. to 75° C. or 68° C. to 72° C. In some embodiments, methods provided herein involve polymerase extension of primers that are annealed to nucleic acid templates at each cycle of a PCR amplification regimen. In some embodiments, a polymerase extension is performed using a polymerase that has relatively strong strand displacement activity. In some embodiments, polymerases having strong strand displacement are useful for preparing nucleic acids for purposes of detecting fusions (e.g., 5′ fusions).

In some embodiments, primer extension is performed under conditions that permit the extension of annealed oligonucleotide primers. As used herein, the term “conditions that permit the extension of an annealed oligonucleotide such that extension products are generated” refers to the set of conditions (e.g., temperature, salt and co-factor concentrations, pH, and enzyme concentration) under which a nucleic acid polymerase catalyzes primer extension. In some embodiments, such conditions are based, at least in part, on the nucleic acid polymerase being used. In some embodiments, a polymerase may perform a primer extension reaction in a suitable reaction preparation. In some embodiments, a suitable reaction preparation contains one or more salts (e.g., 1 to 100 mM KCl, 0.1 to 10 mM MgCl₂), at least one buffering agent (e.g., 1 to 20 mM Tris-HCl), a carrier (e.g., 0.01 to 0.5% BSA), and one or more NTPs (e.g, 10 to 200 μM of each of dATP, dTTP, dCTP, and dGTP). A non-limiting set of conditions is 50 mM KCl, 10 mM Tris-HCl (pH 8.8 at 25° C.), 0.5 to 3 mM MgCl₂, 200 μM each dNTP, and 0.1% BSA at 72° C., under which a polymerase (e.g., Taq polymerase) catalyzes primer extension. In some embodiments, conditions for initiation and extension may include the presence of one, two, three or four different deoxyribonucleoside triphosphates (e.g., selected from dATP, dTTP, dCTP, and dGTP) and a polymerization-inducing agent such as DNA polymerase or reverse transcriptase, in a suitable buffer. In some embodiments, a “buffer” may include solvents (e.g., aqueous solvents) plus appropriate cofactors and reagents which affect pH, ionic strength, etc.

In some embodiments, systems provided herein are configured to implement in an automated fashion multiple nucleic acid amplification cycles. In some embodiments, nucleic acid amplification involve up to 5, up to 10, up to 20, up to 30, up to 40 or more rounds (cycles) of amplification. In some embodiments, nucleic acid amplification may comprise a set of cycles of a PCR amplification regimen from 5 cycles to 20 cycles in length. In some embodiments, an amplification step may comprise a set of cycles of a PCR amplification regimen from 10 cycles to 20 cycles in length. In some embodiments, each amplification step can comprise a set of cycles of a PCR amplification regimen from 12 cycles to 16 cycles in length. In some embodiments, an annealing temperature can be less than 70° C. In some embodiments, an annealing temperature can be less than 72° C. In some embodiments, an annealing temperature can be about 65° C. In some embodiments, an annealing temperature can be from about 61 to about 72° C.

In various embodiments, methods and compositions described herein relate to performing a PCR amplification regimen with one or more of the types of primers described herein. As used herein, “primer” refers to an oligonucleotide capable of specifically annealing to a nucleic acid template and providing a 3′ end that serves as a substrate for a template-dependent polymerase to produce an extension product which is complementary to the template. In some embodiments, a primer is single-stranded, such that the primer and its complement can anneal to form two strands. Primers according to methods and compositions described herein may comprise a hybridization sequence (e.g., a sequence that anneals with a nucleic acid template) that is less than or equal to 300 nucleotides in length, e.g., less than or equal to 300, or 250, or 200, or 150, or 100, or 90, or 80, or 70, or 60, or 50, or 40, or 30 or fewer, or 20 or fewer, or 15 or fewer, but at least 6 nucleotides in length. In some embodiments, a hybridization sequence of a primer may be 6 to 50 nucleotides in length, 6 to 35 nucleotides in length, 6 to 20 nucleotides in length, 10 to 25 nucleotides in length.

Any suitable method may be used for synthesizing oligonucleotides and primers. In some embodiments, commercial sources offer oligonucleotide synthesis services suitable for providing primers for use in methods and compositions described herein (e.g., INVITROGEN™ Custom DNA Oligos (Life Technologies, Grand Island, N.Y.) or custom DNA Oligos from Integrated DNA Technologies (Coralville, Iowa)).

### DNA Shearing/Fragmentation

Nucleic acids used herein (e.g., prior to sequencing) can be sheared, e.g., mechanically or enzymatically sheared, to generate fragments of any desired size. Non-limiting examples of mechanical shearing processes include sonication, nebulization, and AFA™ shearing technology available from Covaris (Woburn, Mass.). In some embodiments, a nucleic acid can be mechanically sheared by sonication. In some embodiments, systems provided here may have one or more vessels, e.g., within a cassette that is fitted within a cartridge, in which nucleic acids are sheared, e.g., mechanically or enzymatically.

In some embodiments, a target nucleic acid is not sheared or digested. In some embodiments, nucleic acid products of preparative steps (e.g., extension products, amplification products) are not sheared or enzymatically digested.

In some embodiments, when a target nucleic acid is RNA, the sample can be subjected to a reverse transcriptase regimen to generate a DNA template and the DNA template can then be sheared. In some embodiments, target RNA can be sheared before performing a reverse transcriptase regimen. In some embodiments, a sample comprising target RNA can be used in methods described herein using total nucleic acids extracted from either fresh or degraded specimens; without the need of genomic DNA removal for cDNA sequencing; without the need of ribosomal RNA depletion for cDNA sequencing; without the need of mechanical or enzymatic shearing in any of the steps; by subjecting the RNA for double-stranded cDNA synthesis using random hexamers.

### Target Nucleic Acid

As used herein, the term “target nucleic acid” refers to a nucleic acid molecule of interest (e.g., a nucleic acid to be analyzed). In some embodiments, a target nucleic acid comprises both a target nucleotide sequence (e.g., a known or predetermined nucleotide sequence) and an adjacent nucleotide sequence which is to be determined (which may be referred to as an unknown sequence). A target nucleic acid can be of any appropriate length. In some embodiments, a target nucleic acid is double-stranded. In some embodiments, the target nucleic acid is DNA. In some embodiments, the target nucleic acid is genomic or chromosomal DNA (gDNA). In some embodiments, the target nucleic acid can be complementary DNA (cDNA). In some embodiments, the target nucleic acid is single-stranded. In some embodiments, the target nucleic acid can be RNA (e.g., mRNA, rRNA, tRNA, long non-coding RNA, microRNA).

In some embodiments, the target nucleic acid can be comprised by genomic DNA. In some embodiments, the target nucleic acid can be comprised by ribonucleic acid (RNA), e.g., mRNA. In some embodiments, the target nucleic acid can be comprised by cDNA. Many of the sequencing methods suitable for use in the methods described herein provide sequencing runs with optimal read lengths of tens to hundreds of nucleotide bases (e.g., Ion Torrent technology can produce read lengths of 200-400 bp). Target nucleic acids comprised, for example, by genomic DNA or mRNA, can be comprised by nucleic acid molecules which are substantially longer than this optimal read length. In order for the amplified nucleic acid portion resulting from the second amplification step to be of a suitable length for use in a particular sequencing technology, the average distance between the known target nucleotide sequence and an end of the target nucleic acid to which the universal oligonucleotide tail-adapter can be ligated should be as close to the optimal read length of the selected technology as possible. For example, if the optimal read-length of a given sequencing technology is 200 bp, then the nucleic acid molecules amplified in accordance with the methods described herein should have an average length of about 400 bp or less. Target nucleic acids comprised by, e.g., genomic DNA or mRNA, can be sheared, e.g., mechanically or enzymatically sheared, to generate fragments of any desired size. Non-limiting examples of mechanical shearing processes include sonication, nebulization, and AFA™ shearing technology available from Covaris (Woburn, Mass.). In some embodiments, a target nucleic acid comprised by genomic DNA can be mechanically sheared by sonication.

In some embodiments, when the target nucleic acid is comprised by RNA, the sample can be subjected to a reverse transcriptase regimen to generate a DNA template and the DNA template can then be sheared. In some embodiments, target RNA can be sheared before performing the reverse transcriptase regimen. In some embodiments, a sample comprising target RNA can be used in the methods described herein using total nucleic acids extracted from either fresh or degraded specimens; without the need of genomic DNA removal for cDNA sequencing; without the need of ribosomal RNA depletion for cDNA sequencing; without the need of mechanical or enzymatic shearing in any of the steps; by subjecting the

RNA for double-stranded cDNA synthesis using random hexamers; and by subjecting the nucleic acid to end-repair, phosphorylation, and adenylation.

In some embodiments, the known target nucleotide sequence can be comprised by a gene rearrangement. The methods described herein are suited for determining the presence and/or identity of a gene rearrangement as the identity of only one half of the gene rearrangement must be previously known (i.e., the half of the gene rearrangement which is to be targeted by the gene-specific primers). In some embodiments, the gene rearrangement can comprise an oncogene. In some embodiments, the gene rearrangement can comprise a fusion oncogene.

As used herein, the term “known target nucleotide sequence” refers to a portion of a target nucleic acid for which the sequence (e.g., the identity and order of the nucleotide bases of the nucleic acid) is known. For example, in some embodiments, a known target nucleotide sequence is a nucleotide sequence of a nucleic acid that is known or that has been determined in advance of an interrogation of an adjacent unknown sequence of the nucleic acid. A known target nucleotide sequence can be of any appropriate length.

In some embodiments, a target nucleotide sequence (e.g., a known target nucleotide sequence) has a length of 10 or more nucleotides, 30 or more nucleotides, 40 or more nucleotides, 50 or more nucleotides, 100 or more nucleotides, 200 or more nucleotides, 300 or more nucleotides, 400 or more nucleotides, 500 or more nucleotides. In some embodiments, a target nucleotide sequence (e.g., a known target nucleotide sequence) has a length in the range of 10 to 100 nucleotides, 10 to 500 nucleotides, 10 to 1000 nucleotides, 100 to 500 nucleotides, 100 to 1000 nucleotides, 500 to 1000 nucleotides, 500 to 5000 nucleotides.

In some embodiments, methods are provided herein for determining sequences of contiguous (or adjacent) portions of a nucleic acid. As used herein, the term “nucleotide sequence contiguous to” refers to a nucleotide sequence of a nucleic acid molecule (e.g., a target nucleic acid) that is immediately upstream or downstream of another nucleotide sequence (e.g., a known nucleotide sequence). In some embodiments, a nucleotide sequence contiguous to a known target nucleotide sequence may be of any appropriate length. In some embodiments, a nucleotide sequence contiguous to a known target nucleotide sequence comprises 1 kb or less of nucleotide sequence, e.g., 1 kb or less of nucleotide sequence, 750 bp or less of nucleotide sequence, 500 bp or less of nucleotide sequence, 400 bp or less of nucleotide sequence, 300 bp or less of nucleotide sequence, 200 bp or less of nucleotide sequence, 100 bp or less of nucleotide sequence. In some embodiments, in which a sample comprises different target nucleic acids comprising a known target nucleotide sequence (e.g., a cell in which a known target nucleotide sequence occurs multiple times in its genome, or on separate, non-identical chromosomes), there may be multiple sequences which comprise “a nucleotide sequence contiguous to” the known target nucleotide sequence. As used herein, the term “determining a (or the) nucleotide sequence,” refers to determining the identity and relative positions of the nucleotide bases of a nucleic acid.

In some embodiments, a known target nucleic acid can contain a fusion sequence resulting from a gene rearrangement. In some embodiments, methods described herein are suited for determining the presence and/or identity of a gene rearrangement. In some embodiments, the identity of one portion of a gene rearrangement is previously known (e.g., the portion of a gene rearrangement that is to be targeted by the gene-specific primers) and the sequence of the other portion may be determined using methods disclosed herein. In some embodiments, a gene rearrangement can involve an oncogene. In some embodiments, a gene rearrangement can comprise a fusion oncogene.

### Samples

In some embodiments, a target nucleic acid is present in or obtained from an appropriate sample (e.g., a food sample, environmental sample, biological sample e.g., blood sample, etc.). In some embodiments, the target nucleic acid is a biological sample obtained from a subject. In some embodiments a sample can be a diagnostic sample obtained from a subject. In some embodiments, a sample can further comprise proteins, cells, fluids, biological fluids, preservatives, and/or other substances. By way of non-limiting example, a sample can be a cheek swab, blood, serum, plasma, sputum, cerebrospinal fluid, urine, tears, alveolar isolates, pleural fluid, pericardial fluid, cyst fluid, tumor tissue, tissue, a biopsy, saliva, an aspirate, or combinations thereof. In some embodiments, a sample can be obtained by resection or biopsy.

In some embodiments, the sample can be obtained from a subject in need of treatment for a disease associated with a genetic alteration, e.g., cancer or a hereditary disease. In some embodiments, a known target sequence is present in a disease-associated gene.

In some embodiments, a sample is obtained from a subject in need of treatment for cancer. In some embodiments, the sample comprises a population of tumor cells, e.g., at least one tumor cell. In some embodiments, the sample comprises a tumor biopsy, including but not limited to, untreated biopsy tissue or treated biopsy tissue (e.g., formalin-fixed and/or paraffin-embedded biopsy tissue).

In some embodiments, the sample is freshly collected. In some embodiments, the sample is stored prior to being used in methods and compositions described herein. In some embodiments, the sample is an untreated sample. As used herein, “untreated sample” refers to a biological sample that has not had any prior sample pre-treatment except for dilution and/or suspension in a solution. In some embodiments, a sample is obtained from a subject and preserved or processed prior to being utilized in methods and compositions described herein. By way of non-limiting example, a sample can be embedded in paraffin wax, refrigerated, or frozen. A frozen sample can be thawed before determining the presence of a nucleic acid according to methods and compositions described herein. In some embodiments, the sample can be a processed or treated sample. Exemplary methods for treating or processing a sample include, but are not limited to, centrifugation, filtration, sonication, homogenization, heating, freezing and thawing, contacting with a preservative (e.g., anti-coagulant or nuclease inhibitor) and any combination thereof. In some embodiments, a sample can be treated with a chemical and/or biological reagent. Chemical and/or biological reagents can be employed to protect and/or maintain the stability of the sample or nucleic acid comprised by the sample during processing and/or storage. In addition, or alternatively, chemical and/or biological reagents can be employed to release nucleic acids from other components of the sample. By way of non-limiting example, a blood sample can be treated with an anti-coagulant prior to being utilized in methods and compositions described herein. Suitable methods and processes for processing, preservation, or treatment of samples for nucleic acid analysis may be used in the method disclosed herein. In some embodiments, a sample can be a clarified fluid sample. In some embodiments, a sample can be clarified by low-speed centrifugation (e.g., 3,000×g or less) and collection of the supernatant comprising the clarified fluid sample.

In some embodiments, a nucleic acid present in a sample can be isolated, enriched, or purified prior to being utilized in methods and compositions described herein. Suitable methods of isolating, enriching, or purifying nucleic acids from a sample may be used. For example, kits for isolation of genomic DNA from various sample types are commercially available (e.g., Catalog Nos. 51104, 51304, 56504, and 56404; Qiagen; Germantown, Md.). In some embodiments, methods described herein relate to methods of enriching for target nucleic acids, e.g., prior to a sequencing of the target nucleic acids. In some embodiments, a sequence of one end of the target nucleic acid to be enriched is not known prior to sequencing. In some embodiments, methods described herein relate to methods of enriching specific nucleotide sequences prior to determining the nucleotide sequence using a next-generation sequencing technology. In some embodiments, methods of enriching specific nucleotide sequences do not comprise hybridization enrichment.

Target Genes (ALK, ROS1, RET) and Therapeutic Applications

In some embodiments of methods described herein, a determination of the sequence contiguous to a known oligonucleotide target sequence can provide information relevant to treatment of disease. Thus, in some embodiments, methods disclosed herein can be used to aid in treating disease. In some embodiments, a sample can be from a subject in need of treatment for a disease associated with a genetic alteration. In some embodiments, a known target sequence is a sequence of a disease-associated gene, e.g., an oncogene. In some embodiments, a sequence contiguous to a known oligonucleotide target sequence and/or the known oligonucleotide target sequence can comprise a mutation or genetic abnormality which is disease-associated, e.g., a SNP, an insertion, a deletion, and/or a gene rearrangement. In some embodiments, a sequence contiguous to a known target sequence and/or a known target sequence present in a sample comprised sequence of a gene rearrangement product. In some embodiments, a gene rearrangement can be an oncogene, e.g., a fusion oncogene.

Certain treatments for cancer are particularly effective against tumors comprising certain oncogenes, e.g., a treatment agent which targets the action or expression of a given fusion oncogene can be effective against tumors comprising that fusion oncogene but not against tumors lacking the fusion oncogene. Methods described herein can facilitate a determination of specific sequences that reveal oncogene status (e.g., mutations, SNPs, and/or rearrangements). In some embodiments, methods described herein can further allow the determination of specific sequences when the sequence of a flanking region is known, e.g., methods described herein can determine the presence and identity of gene rearrangements involving known genes (e.g., oncogenes) in which the precise location and/or rearrangement partner are not known before methods described herein are performed.

In some embodiments, a subject is in need of treatment for lung cancer. In some embodiments, e.g., when the sample is obtained from a subject in need of treatment for lung cancer, the known target sequence can comprise a sequence from a gene selected from the group of ALK, ROS1, and RET. Accordingly, in some embodiments, gene rearrangements result in fusions involving the ALK, ROS1, or RET. Non-limiting examples of gene arrangements involving ALK, ROS1, or RET are described in, e.g., Soda et al. Nature 2007 448561-6: Rikova et al. Cell 2007 131:1190-1203; Kohno et al. Nature Medicine 2012 18:375-7; Takouchi et al. Nature Medicine 2012 18:378-81; which are incorporated by reference herein in their entireties. However, it should be appreciated that the precise location of a gene rearrangement and the identity of the second gene involved in the rearrangement may not be known in advance. Accordingly, in methods described herein, the presence and identity of such rearrangements can be detected without having to know the location of the rearrangement or the identity of the second gene involved in the gene rearrangement.

In some embodiments, the known target sequence can comprise sequence from a gene selected from the group of: ALK, ROS1, and RET.

In some embodiments, the presence of a gene rearrangement of ALK in a sample obtained from a tumor in a subject can indicate that the tumor is susceptible to treatment with a treatment selected from the group consisting of: an ALK inhibitor; crizotinib (PF-02341066); AP26113; LDK378; 3-39; AF802; IPI-504; ASP3026; AP-26113; X-396; GSK-1838705A; CH5424802; diamino and aminopyrimidine inhibitors of ALK kinase activity such as NVP-TAE684 and PF-02341066 (see, e.g., Galkin et al., Proc Natl Acad Sci USA, 2007, 104:270-275; Zou et al., Cancer Res, 2007, 67:4408-4417; Hallberg and Palmer F1000 Med Reports 2011 3:21; Sakamoto et al., Cancer Cell 2011 19:679-690; and molecules disclosed in WO 04/079326). All of the foregoing references are incorporated by reference herein in their entireties. An ALK inhibitor can include any agent that reduces the expression and/or kinase activity of ALK or a portion thereof, including, e.g., oligonucleotides, small molecules, and/or peptides that reduce the expression and/or activity of ALK or a portion thereof. As used herein “anaplastic lymphoma kinase” or “ALK” refers to a transmembrane tyROS line kinase typically involved in neuronal regulation in the wildtype form. The nucleotide sequence of the ALK gene and mRNA are known for a number of species, including human (e.g., as annotated under NCBI Gene ID: 238).

In some embodiments, the presence of a gene rearrangement of ROS1 in a sample obtained from a tumor in a subject can indicate that the tumor is susceptible to treatment with a treatment selected from the group consisting of: a ROS1 inhibitor and an ALK inhibitor as described herein above (e.g., crizotinib). A ROS1 inhibitor can include any agent that reduces the expression and/or kinase activity of ROS1 or a portion thereof, including, e.g., oligonucleotides, small molecules, and/or peptides that reduce the expression and/or activity of ROS1 or a portion thereof. As used herein “c-ros oncogene 1” or “ROS1” (also referred to in the art as ros-1) refers to a transmembrane tyrosine kinase of the sevenless subfamily and which interacts with PTPN6. Nucleotide sequences of the ROS1 gene and mRNA are known for a number of species, including human (e.g., as annotated under NCBI Gene ID: 6098).

In some embodiments, the presence of a gene rearrangement of RET in a sample obtained from a tumor in a subject can indicate that the tumor is susceptible to treatment with a treatment selected from the group consisting of: a RET inhibitor; DP-2490, DP-3636, SU5416; BAY 43-9006, BAY 73-4506 (regorafenib), ZD6474, NVP-AST487, sorafenib, RPI-1, XL184, vandetanib, sunitinib, imatinib, pazopanib, axitinib, motesanib, gefitinib, and withaferin A (see, e.g., Samadi et al., Surgery 2010 148:1228-36; Cuccuru et al., JNCI 2004 13:1006-1014; Akeno-Stuart et al., Cancer Research 2007 67:6956; Grazma et al., J Clin Oncol 2010 28:15s 5559; Mologni et al., J Mol Endocrinol 2006 37:199-212; Calmomagno et al., Journal NCI 2006 98:326-334; Mologni, Curr Med Chem 2011 18:162-175; and the compounds disclosed in WO 06/034833; US Patent Publication 2011/0201598 and U.S. Pat. No. 8,067,434). All of the foregoing references are incorporated by reference herein in their entireties. A RET inhibitor can include any agent that reduces the expression and/or kinase activity of RET or a portion thereof, including, e.g., oligonucleotides, small molecules, and/or peptides that reduce the expression and/or activity of RET or a portion thereof. As used herein, “rearranged during transfection” or “RET” refers to a receptor tyrosine kinase of the cadherin superfamily which is involved in neural crest development and recognizes glial cell line-derived neurotrophic factor family signaling molecules. Nucleotide sequences of the RET gene and mRNA are known for a number of species, including human (e.g., as annotated under NCBI Gene ID: 5979).

Further non-limiting examples of applications of methods described herein include detection of hematological malignancy markers and panels thereof (e.g., including those to detect genomic rearrangements in lymphomas and leukemias), detection of sarcoma-related genomic rearrangements and panels thereof; and detection of IGH/TCR gene rearrangements and panels thereof for lymphoma testing.

In some embodiments, methods described herein relate to treating a subject having or diagnosed as having, e.g., cancer with a treatment for cancer. Subjects having cancer can be identified by a physician using current methods of diagnosing cancer. For example, symptoms and/or complications of lung cancer which characterize these conditions and aid in diagnosis are well known in the art and include but are not limited to, weak breathing, swollen lymph nodes above the collarbone, abnormal sounds in the lungs, dullness when the chest is tapped, and chest pain. Tests that may aid in a diagnosis of, e.g., lung cancer include, but are not limited to, x-rays, blood tests for high levels of certain substances (e.g., calcium), CT scans, and tumor biopsy. A family history of lung cancer, or exposure to risk factors for lung cancer (e.g., smoking or exposure to smoke and/or air pollution) can also aid in determining if a subject is likely to have lung cancer or in making a diagnosis of lung cancer.

Cancer can include, but is not limited to, carcinoma, including adenocarcinoma, lymphoma, blastoma, melanoma, sarcoma, leukemia, squamous cell cancer, small-cell lung cancer, non-small cell lung cancer, gastrointestinal cancer, Hodgkin's and non-Hodgkin's lymphoma, pancreatic cancer, glioblastoma, basal cell carcinoma, biliary tract cancer, bladder cancer, brain cancer including glioblastomas and medulloblastomas; breast cancer, cervical cancer, choriocarcinoma; colon cancer, colorectal cancer, endometrial carcinoma, endometrial cancer; esophageal cancer, gastric cancer; various types of head and neck cancers, intraepithelial neoplasms including Bowen's disease and Paget's disease; hematological neoplasms including acute lymphocytic and myelogenous leukemia; Kaposi's sarcoma, hairy cell leukemia; chronic myelogenous leukemia, AIDS-associated leukemias and adult T-cell leukemia lymphoma; kidney cancer such as renal cell carcinoma, T-cell acute lymphoblastic leukemia/lymphoma, lymphomas including Hodgkin's disease and lymphocytic lymphomas; liver cancer such as hepatic carcinoma and hepatoma, Merkel cell carcinoma, melanoma, multiple myeloma; neuroblastomas; oral cancer including squamous cell carcinoma; ovarian cancer including those arising from epithelial cells, sarcomas including leiomyosarcoma, rhabdomyosarcoma, liposarcoma, fibROS1arcoma, and osteosarcoma; pancreatic cancer; skin cancer including melanoma, stromal cells, germ cells and mesenchymal cells; pROS1tate cancer, rectal cancer; vulval cancer, renal cancer including adenocarcinoma; testicular cancer including germinal tumors such as seminoma, non-seminoma (teratomas, choriocarcinomas), stromal tumors, and germ cell tumors; thyroid cancer including thyroid adenocarcinoma and medullar carcinoma; esophageal cancer, salivary gland carcinoma, and Wilms' tumors. In some embodiments, the cancer can be lung cancer.

### Multiplex Methods

Methods described herein can be employed in a multiplex format. In embodiments of methods described herein, multiplex applications can include determining the nucleotide sequence contiguous to one or more known target nucleotide sequences. As used herein, “multiplex amplification” refers to a process that involves simultaneous amplification of more than one target nucleic acid in one or more reaction vessels. In some embodiments, methods involve subsequent determination of the sequence of the multiplex amplification products using one or more sets of primers. Multiplex can refer to the detection of between about 2-1,000 different target sequences in a single reaction. As used herein, multiplex refers to the detection of any range between 2-1,000, e.g., between 5-500, 25-1,000, or 10-100 different target sequences in a single reaction, etc. The term “multiplex” as applied to PCR implies that there are primers specific for at least two different target sequences in the same PCR reaction.

In some embodiments, target nucleic acids in a sample, or separate portions of a sample, can be amplified with a plurality of primers (e.g., a plurality of first and second target-specific primers). In some embodiments, the plurality of primers (e.g., a plurality of first and second target-specific primers) can be present in a single reaction mixture, e.g., multiple amplification products can be produced in the same reaction mixture. In some embodiments, the plurality of primers (e.g., a plurality of sets of first and second target-specific primers) can specifically anneal to known target sequences comprised by separate genes. In some embodiments, at least two sets of primers (e.g., at least two sets of first and second target-specific primers) can specifically anneal to different portions of a known target sequence. In some embodiments, at least two sets of primers (e.g., at least two sets of first and second target-specific primers) can specifically anneal to different portions of a known target sequence comprised by a single gene. In some embodiments, at least two sets of primers (e.g., at least two sets of first and second target-specific primers) can specifically anneal to different exons of a gene comprising a known target sequence. In some embodiments, the plurality of primers (e.g., first target-specific primers) can comprise identical 5′ tag sequence portions.

In embodiments of methods described herein, multiplex applications can include determining the nucleotide sequence contiguous to one or more known target nucleotide sequences in multiple samples in one sequencing reaction or sequencing run. In some embodiments, multiple samples can be of different origins, e.g., from different tissues and/or different subjects. In such embodiments, primers (e.g., tailed random primers) can further comprise a barcode portion. In some embodiments, a primer (e.g., a tailed random primer) with a unique barcode portion can be added to each sample and ligated to the nucleic acids therein; the samples can subsequently be pooled. In such embodiments, each resulting sequencing read of an amplification product will comprise a barcode that identifies the sample containing the template nucleic acid from which the amplification product is derived.

### Molecular Barcodes

In some embodiments, primers may contain additional sequences such as an identifier sequence (e.g., a barcode, an index), sequencing primer hybridization sequences (e.g., Rd1), and adapter sequences. In some embodiments the adapter sequences are sequences used with a next generation sequencing system. In some embodiments, the adapter sequences are P5 and P7 sequences for Illumina-based sequencing technology. In some embodiments, the adapter sequence are P1 and A compatible with Ion Torrent sequencing technology.

In some embodiments, as used herein, “molecular barcode,” “molecular barcode tag,” and “index” may be used interchangeably, and generally refer to a nucleotide sequence of a nucleic acid that is useful as an identifier, such as, for example, a source identifier, location identifier, date or time identifier (e.g., date or time of sampling or processing), or other identifier of the nucleic acid. In some embodiments, such molecular barcode or index sequences are useful for identifying different aspects of a nucleic acid that is present in a population of nucleic acids. In some embodiments, molecular barcode or index sequences may provide a source or location identifier for a target nucleic acid. For example, a molecular barcode or index sequence may serve to identify a patient from whom a nucleic acid is obtained. In some embodiments, molecular barcode or index sequences enable sequencing of multiple different samples on a single reaction (e.g., performed in a single flow cell). In some embodiments, an index sequence can be used to orientate a sequence imager for purposes of detecting individual sequencing reactions. In some embodiments, a molecular barcode or index sequence may be 2 to 25 nucleotides in length, 2 to 15 nucleotides in length, 2 to 10 nucleotides in length, 2 to 6 nucleotides in length. In some embodiments, a barcode or index comprise at least 2, 3, 4, 5, 6, 7, 8, 9, 10, 11, 12, 13, 14, 15, 16, 17, 18, 19, 20, 21, 22, 23, 24, or at least 25 nucleotides.

In some embodiments, when a population of tailed random primers is used in accordance with methods described herein, multiple distinguishable amplification products can be present after amplification. In some embodiments, because tailed random primers hybridize at various positions throughout nucleic acid molecules of a sample, a set of target-specific primers can hybridize (and amplify) the extension products created by more than 1 hybridization event, e.g., one tailed random primer may hybridize at a first distance (e.g., 100 nucleotides) from a target-specific primer hybridization site, and another tailed random primer can hybridize at a second distance (e.g., 200 nucleotides) from a target-specific primer hybridization site, thereby resulting in two amplification products (e.g., a first amplification product comprising about 100 bp and a second amplification product comprising about 200 bp). In some embodiments, these multiple amplification products can each be sequenced using next generation sequencing technology. In some embodiments, sequencing of these multiple amplification products is advantageous because it provides multiple overlapping sequence reads that can be compared with one another to detect sequence errors introduced during amplification or sequencing processes. In some embodiments, individual amplification products can be aligned and where they differ in the sequence present at a particular base, an artifact or error of PCR and/or sequencing may be present.

### Computer and Control Equipment

The systems provided herein include several components, including sensors, environmental control systems (e.g., heaters, fans), robotics (e.g., an XY positioner), etc. which may operate together at the direction of a computer, processor, microcontroller or other controller. The components may include, for example, an XY positioner, a liquid handling devices, microfluidic pumps, linear actuators, valve drivers, a door operation system, an optics assembly, barcode scanners, imaging or detection system, touchscreen interface, etc.

In some cases, operations such as controlling operations of a systems and/or components provided therein or interfacing therewith may be implemented using hardware, software or a combination thereof. When implemented in software, the software code can be executed on any suitable processor or collection of processors, whether provided in a single component or distributed among multiple components. Such processors may be implemented as integrated circuits, with one or more processors in an integrated circuit component. A processor may be implemented using circuitry in any suitable format.

A computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable, mobile or fixed electronic device, including the system itself.

In some cases, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. In other examples, a computer may receive input information through speech recognition or in other audible format, through visible gestures, through haptic input (e.g., including vibrations, tactile and/or other forces), or any combination thereof.

One or more computers may be interconnected by one or more networks in any suitable form, including as a local area network or a wide area network, such as an enterprise network or the Internet. Such networks may be based on any suitable technology and may operate according to any suitable protocol and may include wireless networks, wired networks, or fiber optic networks.

The various methods or processes outlined herein may be coded as software that is executable on one or more processors that employ any one of a variety of operating systems or platforms. Such software may be written using any of a number of suitable programming languages and/or programming or scripting tools, and may be compiled as executable machine language code or intermediate code that is executed on a framework or virtual machine.

One or more algorithms for controlling methods or processes provided herein may be embodied as a readable storage medium (or multiple readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other tangible storage medium) encoded with one or more programs that, when executed on one or more computers or other processors, perform methods that implement the various methods or processes described herein.

In some embodiments, a computer readable storage medium may retain information for a sufficient time to provide computer-executable instructions in a non-transitory form. Such a computer readable storage medium or media can be transportable, such that the program or programs stored thereon can be loaded onto one or more different computers or other processors to implement various aspects of the methods or processes described herein. As used herein, the term “computer-readable storage medium” encompasses only a computer-readable medium that can be considered to be a manufacture (e.g., article of manufacture) or a machine. Alternatively or additionally, methods or processes described herein may be embodied as a computer readable medium other than a computer-readable storage medium, such as a propagating signal.

The terms “program” or “software” are used herein in a generic sense to refer to any type of code or set of executable instructions that can be employed to program a computer or other processor to implement various aspects of the methods or processes described herein. Additionally, it should be appreciated that according to one aspect of this embodiment, one or more programs that when executed perform a method or process described herein need not reside on a single computer or processor, but may be distributed in a modular fashion amongst a number of different computers or processors to implement various procedures or operations.

Executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. Non-limiting examples of data storage include structured, unstructured, localized, distributed, short-term and/or long term storage. Non-limiting examples of protocols that can be used for communicating data include proprietary and/or industry standard protocols (e.g., HTTP, HTML, XML, JSON, SQL, web services, text, spreadsheets, etc., or any combination thereof). For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags, or other mechanisms that establish relationship between data elements.

In some embodiments, information related to the operation of the system (e.g., temperature, imaging or optical information, fluorescent signals, component positions (e.g., heated lid position, rotary valve position), liquid handling status, barcode status, bay access door position or any combination thereof) can be obtained from one or more sensors or readers associated with the system (e.g., located within the system), and can be stored in computer-readable media to provide information about conditions during a process (e.g., an automated library preparation process). In some embodiments, the readable media comprises a database. In some embodiments, said database contains data from a single system (e.g., from one or more bays). In some embodiments, said database contains data from a plurality of systems. In some embodiments, data is stored in a manner that makes it tamper-proof. In some embodiments, all data generated by the system is stored. In some embodiments, a subset of data is stored.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Any terms as used herein related to shape, orientation, alignment, and/or geometric relationship of or between, for example, one or more articles, structures, forces, fields, flows, directions/trajectories, and/or subcomponents thereof and/or combinations thereof and/or any other tangible or intangible elements not listed above amenable to characterization by such terms, unless otherwise defined or indicated, shall be understood to not require absolute conformance to a mathematical definition of such term, but, rather, shall be understood to indicate conformance to the mathematical definition of such term to the extent possible for the subject matter so characterized as would be understood by one skilled in the art most closely related to such subject matter. Examples of such terms related to shape, orientation, and/or geometric relationship include, but are not limited to terms descriptive of: shape—such as, round, square, circular/circle, rectangular/rectangle, triangular/triangle, cylindrical/cylinder, elliptical/ellipse, (n)polygonal/(n)polygon, etc.; angular orientation—such as perpendicular, orthogonal, parallel, vertical, horizontal, collinear, etc.; contour and/or trajectory—such as, plane/planar, coplanar, hemispherical, semi-hemispherical, line/linear, hyperbolic, parabolic, flat, curved, straight, arcuate, sinusoidal, tangent/tangential, etc.; direction—such as, north, south, east, west, etc.; surface and/or bulk material properties and/or spatial/temporal resolution and/or distribution—such as, smooth, reflective, transparent, clear, opaque, rigid, impermeable, uniform(ly), inert, non-wettable, insoluble, steady, invariant, constant, homogeneous, etc.; as well as many others that would be apparent to those skilled in the relevant arts. As one example, a fabricated article that would described herein as being “square” would not require such article to have faces or sides that are perfectly planar or linear and that intersect at angles of exactly 90 degrees (indeed, such an article can only exist as a mathematical abstraction), but rather, the shape of such article should be interpreted as approximating a “square,” as defined mathematically, to an extent typically achievable and achieved for the recited fabrication technique as would be understood by those skilled in the art or as specifically described. As another example, two or more fabricated articles that would described herein as being “aligned” would not require such articles to have faces or sides that are perfectly aligned (indeed, such an article can only exist as a mathematical abstraction), but rather, the arrangement of such articles should be interpreted as approximating “aligned,” as defined mathematically, to an extent typically achievable and achieved for the recited fabrication technique as would be understood by those skilled in the art or as specifically described.

What is claimed:

1. An apparatus for performing a chemical process, the apparatus comprising:
a vessel;
a magnetic assembly positioned adjacent to the vessel, the magnetic assembly comprising one or more retractable magnets, each of the one or more retractable magnets capable of moving between i) a deployed position that is sufficiently proximate to a plurality of magnetic particles disposed in the vessel to force a magnetic particle present in the vessel against a wall of the vessel and ii) a retracted position.

2. The apparatus of claim 1, further comprising one or more actuators configured to translocate the one or more retractable magnets between the deployed position and the retracted position.

3. The apparatus of claim 1, further comprising a thermal assembly positioned adjacent to the vessel, the thermal assembly comprising one or more thermal elements configured to heat or cool the vessel.

4. The apparatus of claim 3, wherein each thermal element is a thermal pin defining a hollow portion in which one of the one or more retractable magnets is positioned.

5. The apparatus of claim 1, wherein the magnetic assembly comprises 2-24 retractable magnets.

6. The apparatus of claim 5, wherein the magnetic assembly comprises 6 retractable magnets.

7. The apparatus of claim 1, wherein the one or more retractable magnets are independently-controllable.

8. The apparatus of any prior claim, wherein the chemical process is a nucleic acid purification.

9. The apparatus of any prior claim, further comprising a sample fluid disposed in the vessel, the sample fluid comprising a plurality of magnetic particles having bound molecules of interest.

10. The apparatus of claim 9, wherein the bound molecules of interest are selected from the group consisting of nucleic acid molecules and proteins.

11. The apparatus of claim 9, wherein the plurality of magnetic particles comprises a plurality of magnetic beads.

12. An apparatus for performing a chemical process, the apparatus comprising:
a vessel;
a sample fluid disposed in the vessel, the sample fluid comprising a plurality of magnetic beads having bound nucleic acid;
a magnetic assembly positioned adjacent to the vessel, the magnetic assembly comprising:
one or more magnets;
one or more independently-controllable linear actuators coupled to the one or more magnets, each of the one or more independently-controllable linear actuators capable of moving the one or more magnets to a deployed position sufficiently proximate to the plurality of magnetic beads to draw the plurality of magnetic beads to a wall of the vessel, and a retracted position; and
one or more magnet lifting apparatuses coupled to the one or more magnets, each of the one or more magnet lifting apparatuses capable of moving the one or more magnets to an extended position and a contracted position; and
a heating assembly positioned adjacent to the vessel, the heating assembly comprising:
one or more heating pins, wherein each of the heating pins defines a hollow portion in which one of the one or more magnets is positioned;
a heating block defining one or more through holes, each through hole positioned to receive one of the one or more heating pins therethrough;
an insulating liner positioned to surround at least a portion of the heating block, the insulating liner defining one or more through holes, each through hole positioned to receive one of the one or more heating pins therethrough;
a cartridge heater disposed proximate to the heating block; and
a thermistor disposed proximate to the heating block.

13. A method for performing a chemical process, the method comprising:
introducing a sample fluid containing a nucleic acid to a vessel;
introducing a plurality of magnetic beads to the sample fluid in the vessel;
mixing the sample fluid to form a homogenous mixture comprising the plurality of magnetic beads with bound nucleic acid and a remainder portion;
deploying one or more retractable magnets into a position sufficiently proximate to the plurality of magnetic beads to draw the plurality of magnetic beads with bound nucleic acid to a wall of the vessel; and
removing the remainder portion from the vessel.

14. The method of claim 13, further comprising rinsing the nucleic acid that is bound to the magnetic beads with a solvent.

15. The method of claim 13 or 14, further comprising retracting the one or more retractable magnets into a position sufficiently distant from the plurality of magnetic beads to release the plurality of magnetic beads from the wall of the vessel.

16. The method of any of claims 13-15, further comprising introducing elution buffer into the vessel to release the nucleic acid from the magnetic beads.

17. The method of any of claims 13-16, further comprising deploying one or more retractable magnets into a position sufficiently proximate to the plurality of magnetic beads to draw the plurality of magnetic beads to the wall of the vessel to provide a purified solution containing nucleic acid; and
removing the purified solution containing nucleic acid from the vessel.

18. The method of claim 14, wherein the solvent is ethanol.
